(19) United States
(12) Patent Application Publication
(10) Pub. No.: US 2023/0230672 A1
(43) Pub. Date: Jul. 20, 2023

(54) SYSTEM AND METHOD FOR PROVIDING FERTILITY ENHANCING DIETARY RECOMMENDATIONS IN INDIVIDUALS WITH PROSTATE CONDITIONS

(71) Applicant: SOCIÉTÉ DES PRODUITS NESTLÉ S.A., Vevey (CH)

(72) Inventors: MYRIAM AFEICHE ZEHIL, Lausanne 26 (CH); FLORENT DUDAN, St-Sulpice (CH); FABIO MAINARDI, Lausanne (CH); ROKO PLESTINA, Morges (CH)

(21) Appl. No.: 17/997,278

(22) PCT Filed: Apr. 27, 2021
(86) PCT No.: PCT/EP2021/060887

§ 371 Date: Oct. 27, 2022

(30) Foreign Application Priority Data
Apr. 29, 2020 (EP) 20172020.8

(51) Int. Cl. G16H 20/60

(52) U.S. Cl. CPC G16H 20/60

(57) ABSTRACT

The present invention presents new and innovative methods and systems for personalized, real-time diet and lifestyle recommendations for users that are seeking to improve their own fertility. In a preferred embodiment, the present invention relates to novel methods and systems for personalized dietary recommendations for improving fertility in individuals, especially men, with prostate conditions.

### FIELD OF THE INVENTION

The present invention presents new and innovative methods and systems for personalized, real-time diet and lifestyle recommendations for users that are seeking to improve their own fertility.

In a preferred embodiment, the present invention relates to novel methods and systems for personalized dietary recommendations for improving fertility in individuals, especially men, with prostate conditions.

### BACKGROUND TO THE INVENTION

The prostate is the target of a number of common diseases and conditions that can affect male fertility at different ages. In both young and aged men, prostatic diseases or an unhealthy prostate can affect spermatozoa production and fertility.

The prostate gland produces fluid that makes up the semen and contains sperm. The prostate surrounds the urethra that carries urine away from the bladder and out of the body. With increasing age, the prostate gradually hypertrophies and may lead to prostate problems.

Some common problems are: prostatitis, benign prostatic hyperplasia (BPH) and recovery after prostate surgery or radiation.

### Prostatitis

Prostatis is an inflammation of the prostate, usually caused by bacteria. Treatment depends on the type of prostatitis. Bacterial prostatitis is treated with antibiotics. For non-bacterial, chronic prostatitis, treatment includes pain, discomfort, and inflammation management; alpha-blocker to relax prostate and bladder muscles; 5-alpha reductase inhibitors, anti-inflammatories (nonsteroidal anti-inflammatory drugs (NSAIDs), corticosteroids), relaxation exercises, and physical therapy.

### Benign Prostatic Hyperplasia (BPH)

BPH is characterized by the appearance of hyperplastic nodules found primarily in the peri-urethral region and transition zone of the prostate where it enlarges and starts impinging on the urethra. This results in a constellation of symptoms called lower urinary tract symptoms (LUTS). These include irritative voiding symptoms such as frequency, urgency and nocturia, and obstructive symptoms such as weak or intermittent urinary stream, incomplete bladder emptying, and straining to void.

Treatments for BPH include watchful waiting, lifestyle changes such as drinking less liquids before going to bed; medicines to stop prostate growth or shrink the prostate; an alpha-blocker or other medicine that relaxes prostate and bladder muscles; and surgery.

### Recovery after Prostate Surgery or Radiation

Men treated for prostate cancer, by surgery or radiation, are at a heightened risk of infertility. Infertility risk depends on the extent of the prostatectomy that removes all or part of the prostate gland and the seminal vesicles. The seminal vesicles help the prostate to deliver semen through the urethra and ejaculate it out of the penis. If both the prostate and seminal vesicles are resected then there is no chance for the sperm to be delivered outside the penis and the man becomes infertile. However, if the seminal vesicles and the prostate are preserved then it may be possible to remain fertile. A fertility specialist may recommend freezing semen before the surgery or radiation.

As men are delaying the age at which they would like to remain fertile, they are at increasing risk of developing prostate diseases and conditions. This is exacerbated by obesity which contributes to lower urinary tract symptoms (LUTS) as well. Therefore, there is a need to find ways to improve fertility in men at risk of having prostate conditions.

At present, dietary recommendations for men with prostate conditions are often too general as studies on fertility and diet often investigate only single nutrients at a time or food groups without putting them in the context of an entire diet over a day or entire meal and they do not provide recommended intake amounts to be consumed per day or for each specific, different medical condition which affects fertility, such as prostate conditions.

The present invention addresses the deficiencies in the state of the art by providing new and innovative methods and systems for personalized, real-time diet and lifestyle recommendations for individual users.

The dietary recommendations of the present invention may contribute to improving prostate-related symptoms such as inflammation of the prostate and lower urinary tract symptoms (LUTS).

In particular, the present invention addresses the specific condition of enhancing fertility and conception in individuals with prostate conditions, by providing a novel, consolidated dietary recommendations which combine:

specific dietary components recommended to be consumed daily

specific dietary intake amounts

specific recommendations on avoiding certain dietary components

specific recommendations on lifestyle components.

### SUMMARY OF THE INVENTION

The present invention presents novel and innovative methods and systems for personalized, real-time diet and lifestyle recommendations for users that are seeking to improve their own fertility.

In several embodiments, a method and system is provided that includes requesting and receiving a plurality of user attributes, comparing the plurality of user attributes to a corresponding plurality of evidence-based fertility benchmarks, determining a plurality of fertility support opportunities based on the plurality of user attributes and the comparison to the corresponding plurality of evidence-based fertility benchmarks, identifying a plurality of fertility enhancing recommendations based on the plurality of fertility support opportunities, and presenting at least one of the plurality of fertility enhancing recommendations.

In a preferred embodiment of the invention, the system and method present fertility enhancing recommendations are for increasing fertility in a male individual with prostate conditions such as prostatitis, benign prostate hyperplasia (BHP) and recovery after prostate surgery or radiation.

In another preferred embodiment of the invention, the dietary recommendations of the present invention may contribute to improving prostate-related symptoms such as inflammation of the prostate or lower urinary tract symptoms (LUTS).

### DESCRIPTION OF FIGURES

FIG. 1 illustrates a system according to an embodiment of the present invention.

FIG. 2 illustrates system components according to exemplary embodiments of the present invention.

FIG. 3 illustrates system components according to an exemplary embodiment of the present invention.

FIG. 4 illustrates an example plurality of dietary recommendations for a user according to an exemplary embodiment of the present invention.

FIG. 5 illustrates a method according to an embodiment of the present invention.

FIGS. 6A and 6B illustrate a method according to an embodiment of the present invention.

### DETAILED DESCRIPTION OF THE INVENTION

To promote user fertility, it may be useful to provide a customized diet and lifestyle plan for users interested enhancing fertility. Therefore, a customized, integrated approach is necessary to provide maximum benefit to enhance chances of fertility and conception.

In several embodiments of the invention, the customized diet and lifestyle plan is individualized to male users with prostate conditions.

In a preferred embodiment of the invention, the customized diet and lifestyle plan is individualized to male users with prostate conditions, such as prostatitis, benign prostatic hyperplasia (BPH) and recovery after prostate surgery or radiation.

In a preferred embodiment of the invention, the customized diet and lifestyle plan is individualized to male users with prostate conditions such as inflammation of the prostate and lower urinary tract symptoms.

In any of the above conditions of prostate conditions, the methods and systems of the invention for providing individualized recommendations may be beneficial.

In several embodiments of the invention, the recommendations are personalized based on information from an individual regarding certain medical conditions or diseases and the current status of conception to compare with a historical evidence-based fertility database to generate recommended diet and lifestyle options that will help improve the patient's fertility based on the provided information.

An example system may be beneficial if it can provide user support throughout the entire conception journey, from the early stages of planning to the final stages of conception. As such, this example system would be useful should it provide constant, around the clock access to both virtual and personal fertility, lifestyle, nutrition, and exercise coaches. Furthermore, an example system may provide recommendations to manage anxiety, reduce stress, or provide specific supplementation, all of which are also linked to a user's fertility. The system may provide different recommendations for the male and female individuals in the couple that wish to conceive.

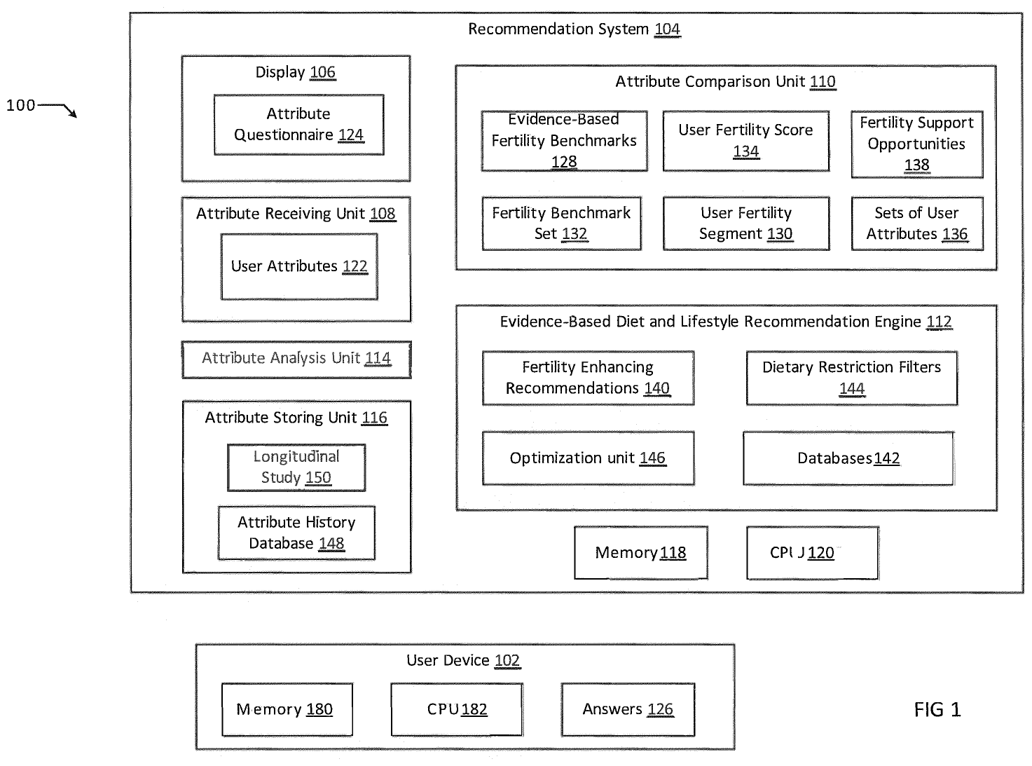

FIG. 1 illustrates a system 100 according to an embodiment of the present disclosure. The system 100 includes a user device 102 and a recommendation system 104. The user device 102 may be implemented as a computing device, such as a computer, smartphone, tablet, smartwatch, or other wearable through which an associated user can communicate with the recommendation system 104. The user device 102 may also be implemented as, e.g., a voice assistant configured to receive voice requests from a user and to process the requests either locally on a computer device proximate to the user or on a remote computing device (e.g., at a remote computing server).

The recommendation system 104 includes one or more of a display 106, an attribute receiving unit 108, an attribute comparison unit 110, an evidence-based diet and lifestyle recommendation engine 112, an attribute analysis unit 114, an attribute storing unit 116, a memory 118, and a CPU 120. Note, that in some embodiments, a display 106 may additionally or alternatively be located within the user device 102. In an example, the recommendation system 104 may be configured to receive a request for a plurality of fertility enhancing recommendations 140. For example, a user may install an application on the user device 102 that requires the user to sign up for a recommendation service. By signing up for the service, the user device 102 may send a request for the fertility enhancing recommendations 140. In a different example, the user may use the user device 102 to access a web portal using user-specific credentials. Through this web portal, the user may cause the user device 102 to request fertility enhancing recommendations from the recommendation system 104.

In another example, the recommendation system 104 may be configured to request and receive a plurality of user attributes 122. For example, the display 106 may be configured to present an attribute questionnaire 124 to the user. The attribute receiving unit 108 may be configured to receive the user attributes 122. In one example, the attribute receiving unit 108 may receive a plurality of answers 126 based on the attribute questionnaire 124, and based on the plurality of answers, determine the plurality of user attributes 122. For example, the attribute receiving unit 108 may receive answers to the attribute questionnaire 124 suggesting that the diet of the user is equivalent to the recommended dietary allowance (“RDA”) and then determine the user attributes 122 to be equivalent to the RDA. In another example, the user device attribute receiving unit 108 may directly receive the user attributes 122 from the user device 102.

In another example, the attribute receiving unit 108 may be configured to receive the test results of a home-test kit, the results of a standardized health test administered by a medical professional, the results of a self-assessment tool used by the user, or the results of any external or third party test. Based on the results from any of these tests or tools, the attribute receiving unit 108 may be configured to determine the user attributes 122. For example, this may be measurements of the nutrient levels in blood or urine of the user which may be compared to standardized nutrient levels.

The recommendation system 104 may be further configured to compare the plurality of user attributes 122 to a corresponding plurality of evidence-based fertility benchmarks 128. For example, the attribute comparison unit 110 may be configured to determine a user fertility segment 130.

In a preferred example, the user fertility segment could be much more specific. For example, the user fertility segment could be a user with prostate conditions.

Furthermore, the attribute comparison unit 110 may be further configured to determine a fertility benchmark set 132 based on the user fertility segment 130. For example, if the attribute comparison unit 110 determines that a user falls into the prostate condition user fertility segment 130, based on the plurality of user attributes 122, the attribute comparison unit 110 may select a fertility benchmark set 132 that has been created and defined according to the specific needs of a user undergoing a particular medical treatment, such as hormonal treatment.

The comparison unit 110 may be further configured to select, from this determined fertility benchmark set 132, the evidence-based fertility benchmarks 128 and compare the now selected evidence-based fertility benchmarks 128 to each of the corresponding user attributes 122. For example, when the fertility benchmark set 132 has been determined, in response to the determination, the attribute comparison unit 110 may compare a user attribute 122 that represents the user's vitamin D intake to an evidence based fertility benchmark 128 that represents a benchmark vitamin D intake, determining whether the user is below, at, or above the benchmark vitamin D intake. Though this example is based on a concrete, numerical comparison, another example of a benchmark comparison may be qualitative and different depending on a person. For example, a user attribute 122 may indicate that the user is currently experiencing higher than normal levels of stress. An example benchmark related to a user stress level may indicate that an average or low level of stress is desired and thus, the user attribute 122 indicating a higher level of stress is determined to be below that of the benchmark. As different users experience differing levels of stress, even under the same circumstances, such a comparison requires a customized approach.

In addition, during the comparison from the prior example, the attribute comparison unit 110 may be configured to determine a user fertility score 134 based on the comparison between the evidence-based fertility benchmarks 128 and the user attributes 122. For example, the attribute comparison unit 110 may determine a user fertility score of 95/100 if the user attributes 122 very nearly meet all or most of the corresponding evidence-based fertility benchmarks 128. In another example, a score may be represented through lettering grades, symbols, or any other system of ranking that allows a user to interpret how well their current attributes rate amongst benchmarks. This user fertility score 134 may be presented through the display 106.

The recommendation system 104 may be further configured to determine a plurality of fertility support opportunities 138 based on the plurality of user attributes 122 and the comparison to the corresponding plurality of evidence-based fertility benchmarks 128. In one example, the attribute comparison unit 110 may determine fertility support opportunities 138 for every user attribute 122 that does not meet the corresponding evidence-based fertility benchmark. In this example, a corresponding evidence-based fertility benchmark 128 may require a user have an intake of 1000 mg/day of Vitamin D, whereas the user attribute may indicate the user is only receiving 500 mg/day of Vitamin D. Therefore, the attribute comparison unit 110 may determine an increase in Vitamin D intake to be a fertility support opportunity 138.

In another example, the attribute comparison unit 110 may be configured to identify a first set of user attributes 136 comprised of each of the plurality of user attributes 122 that are below the corresponding one of the plurality of evidence-based fertility benchmarks 128 as well as identify a second set of user attributes 136 comprised of each of the plurality of user attributes 122 that are greater than or equal to the corresponding evidence-based fertility benchmarks 128. While the first set of user attributes 136 is determined similarly to the above given example, the second set of user attributes 136 differs in that, although the associated user does not appear to have a deficiency, there may be opportunities to support fertility by recommending the user maintain current practices or opportunities to further improve upon them. Accordingly, the recommendation system 104 may determine opportunities to support fertility based on which attributes 122 populate either sets 136.

The recommendation system 104 may be further configured to identify a plurality of fertility enhancing recommendations 140 based on the plurality of fertility support opportunities 138. For example, the evidence-based diet and lifestyle recommendation engine 112 may be configured to be cloud-based. The recommendation engine 112 may comprise one or more of a plurality of databases 142, a plurality of dietary restriction filters 144, and an optimization unit 146. Based on the plurality of opportunities 138, the recommendation engine 112 may identify the plurality of fertility enhancing recommendations 140 according to the one or more of plurality of databases 142, the dietary restriction filters 144, and the optimization unit 146.

In another example, the recommendation system 104 may be configured to provide continuous recommendations, based on prior user attributes. For example, the recommendation system 104 may comprise, in addition to the previously discussed elements, an attribute storing unit 116 and an attribute analysis unit 114. The attribute storing unit 116 may be configured to, responsive to the attribute receiving unit 108 receiving the plurality of user attributes 122, add the received user attributes 122 to an attribute history database 148 as a new entry based on when the plurality of user attributes 122 were received. For example, if user attributes 122 are received by the attribute receiving unit 108 on a first day, the attribute storing unit 116 will add the received user attributes 122 to a cumulative attribute history database 148 noting the date of entry, in this case the first day. Later, if user attributes 122 are received by the attribute receiving unit 108 on a second day, e.g. the next day, the attribute storing unit 116 will also add these new attributes to the attribute history database 148, noting that they were received on the second day, while also preserving the earlier attributes from the first day.

This attribute analysis unit 114 may be configured to analyze the plurality of user attributes 122 stored within the attribute history database 148, wherein analyzing the stored plurality of user attributes 122 comprises performing a longitudinal study 150. Continuing the earlier example, the attribute analysis unit 114 may perform a longitudinal study of the user attributes 122 from each of the first day, the second day, and every other collection of user attributes 122 found within the attribute history database 148. The evidence based diet and lifestyle recommendation engine 112 may be further configured to generate a plurality of fertility enhancing recommendations 140 based on at least the stored user attributes 122 found within the attribute history database 148 and the analysis performed by the attribute analysis unit 114.

In an embodiment, the attribute analysis unit 114 is further configured to repeatedly analyze the plurality of user attributes 122 stored within the attribute history database 148 responsive to the attribute storing unit 116 adding a new entry to the attribute history database 148, essentially re-analyzing all of the data within the attribute history database 148 immediately after new user attributes 122 are received. Similarly, the evidence based diet and lifestyle recommendation engine 112 may be further configured to repeatedly generate the plurality of fertility enhancing recommendations 140 responsive to the attribute analysis unit 114 completing an analysis, thereby effectively generating new fertility enhancing recommendations 140 that consider all past and present user attributes 122 each time a new set of user attributes 122 is received.

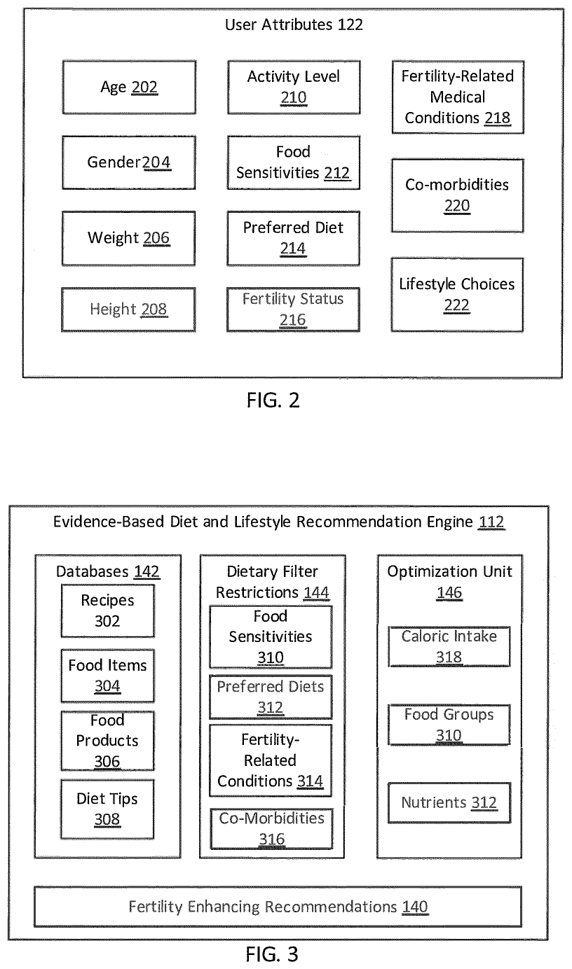

FIG. 2 illustrates an example database containing a plurality of user attributes 122. For example, the user attributes 122 may be populated by information regarding one or more of age 202, gender 204, weight 206, height 208, activity level 210, food sensitivities 212, preferred diet 214, fertility status 216, fertility-related medical conditions 218, co-morbidities 220, and lifestyle choices 222. Some examples of food sensitivities 212 include lactose, eggs, nuts, shellfish, soy, fish, and gluten sensitivities. Some non-limiting examples of a preferred diet 214 includes vegetarian, vegan, Mediterranean, kosher, halal, paleo, low carb, and low fat diets.

Some non-limiting examples of fertility-related medical conditions 218 include prostate conditions, polycystic ovary syndrome, premature ovarian insufficiency, endometriosis, recurring pregnancy loss, undergoing IVF, semen abnormality, misuse of anabolic steroids and protein supplements, erectile dysfunction, hormonal imbalance, low testosterone, and prostate issues.

In a preferred embodiment, the fertility-related medical condition 218 is a prostate related condition such as prostatitis, benign prostate hyperplasia (BPH) or recovery after prostate surgery or radiation.

Some non-limiting examples of co-morbidities 220 include diabetes, obesity, high blood pressure, high cholesterol, celiac, and heartburn. Some non-limiting examples off lifestyle choices 222 may include sleeping habits such as the typical hours of sleep per night, stress attributes such as the level of stress currently experienced by the user or typical levels of stress experienced, whether the user smokes, the number of alcoholic drinks typically consumed, exercise frequency, or any other lifestyle choices 222 that may have a bearing on fertility.

FIG. 3 illustrates an example embodiment of an evidence-based diet and lifestyle recommendation engine 112. In an example embodiment, the evidence-based diet and lifestyle recommendation engine 112 comprises a plurality of databases 142, a plurality of dietary filter restrictions 144, and an optimization unit 146. The plurality of databases 142 may include a database comprised of one or more of recipes 302, food items 304, food products 306, and diet tips 308. The dietary filter restrictions 144 may comprise filters for one or more of food sensitivities 310, preferred diets 312, fertility-related conditions 314, and co-morbidities 316. The optimization unit 146 may contain optimization rules based on one or more of caloric intake 318, food groups 310, and specific nutrients 312.

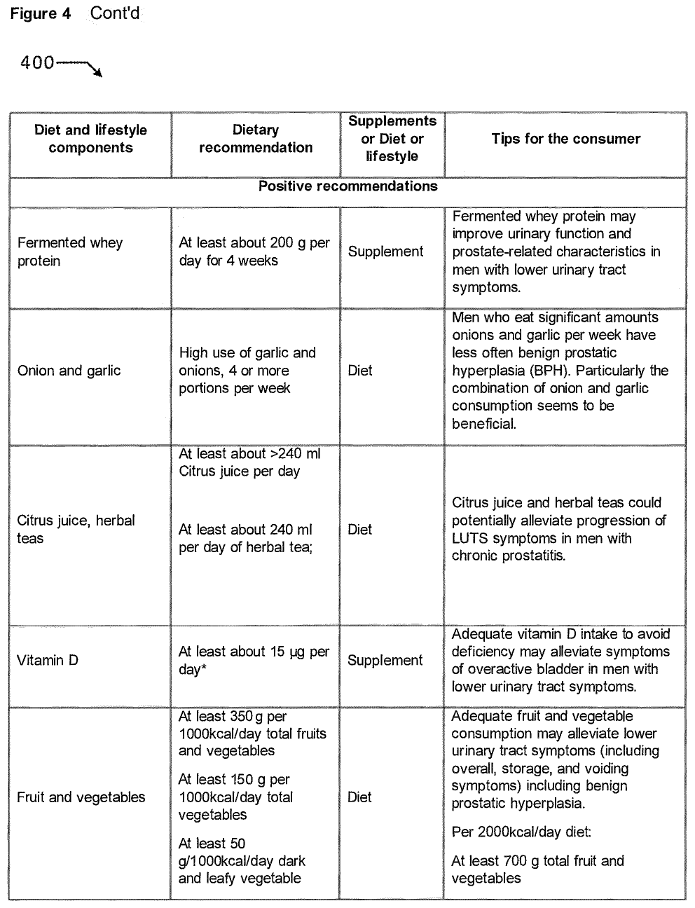

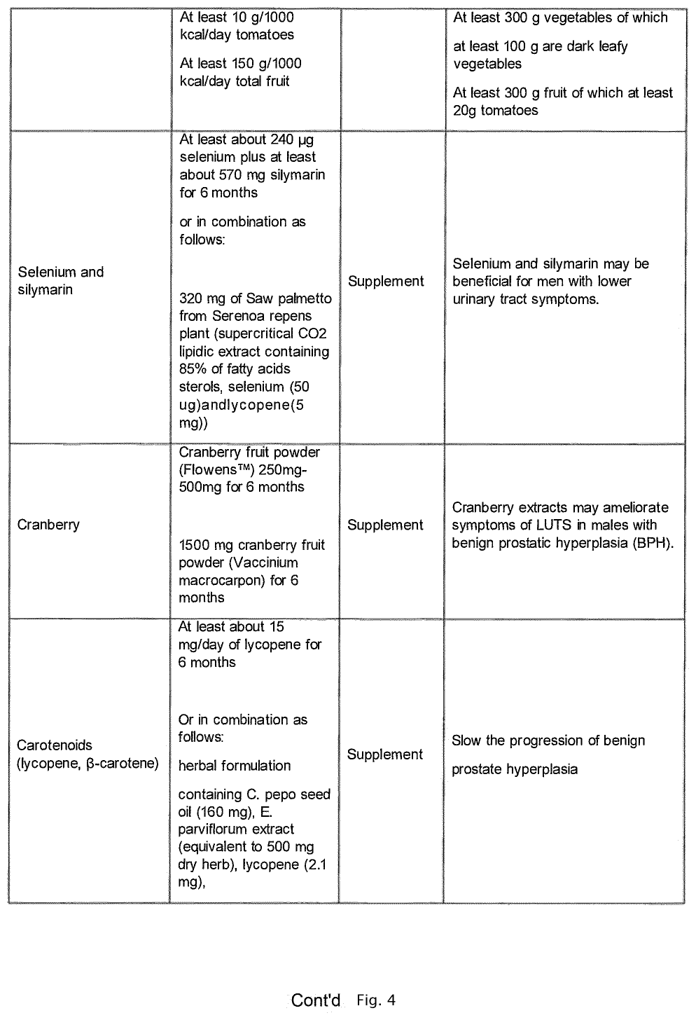

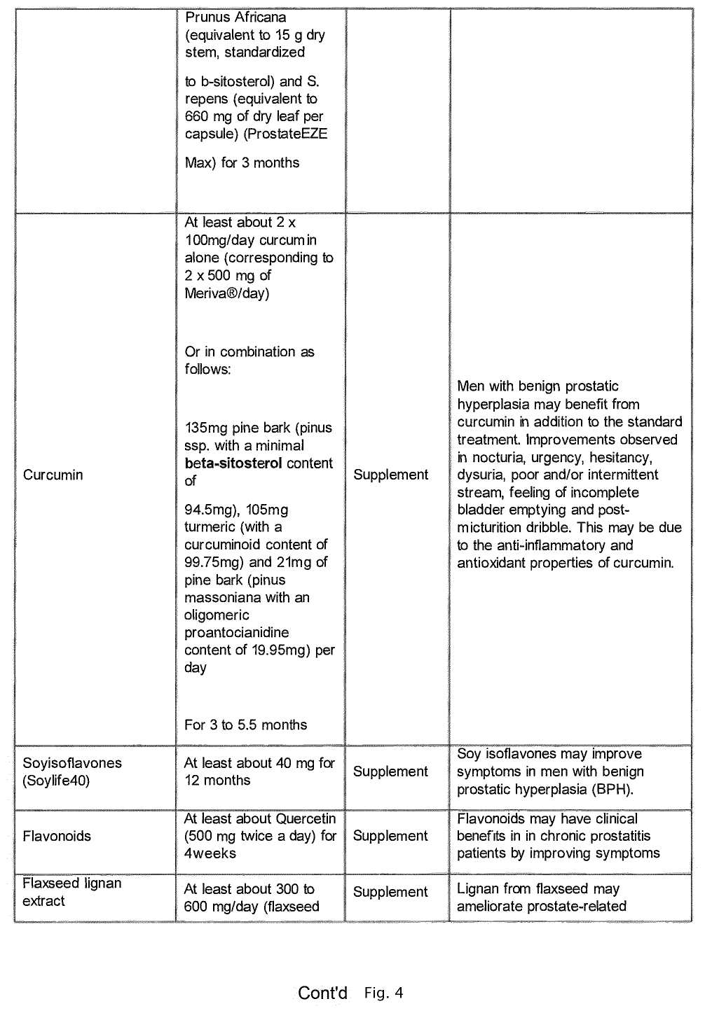

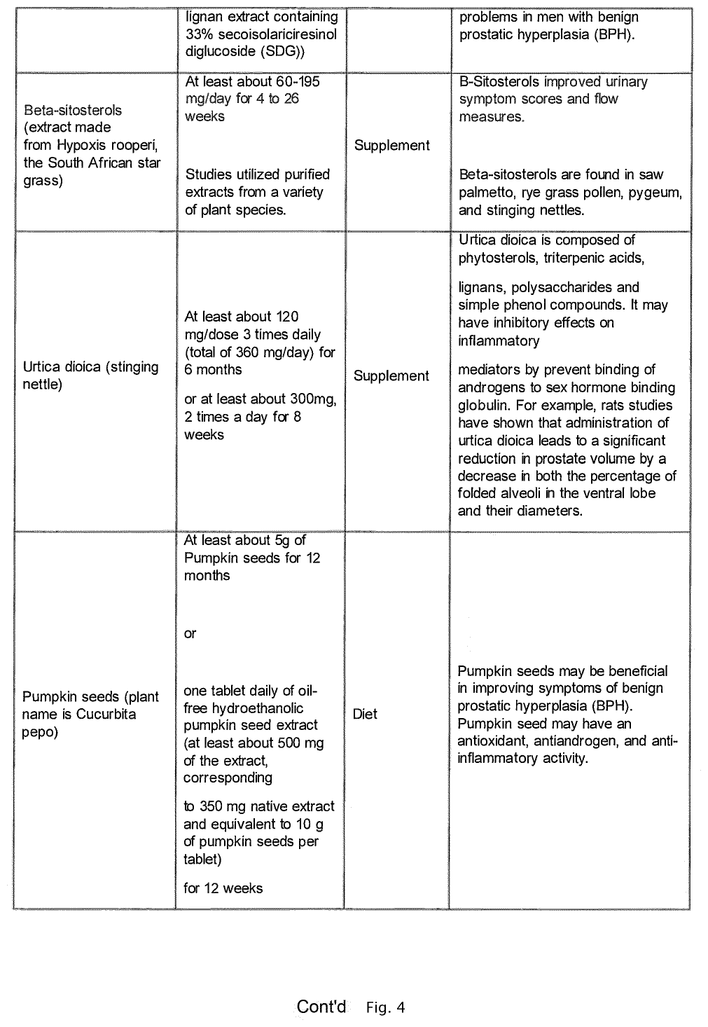

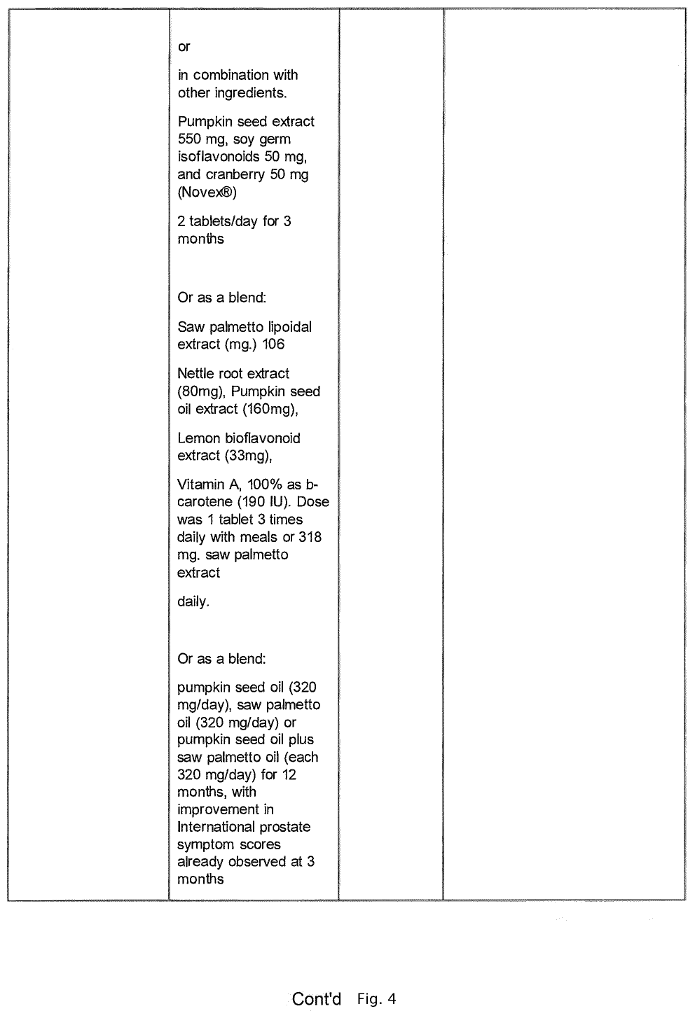

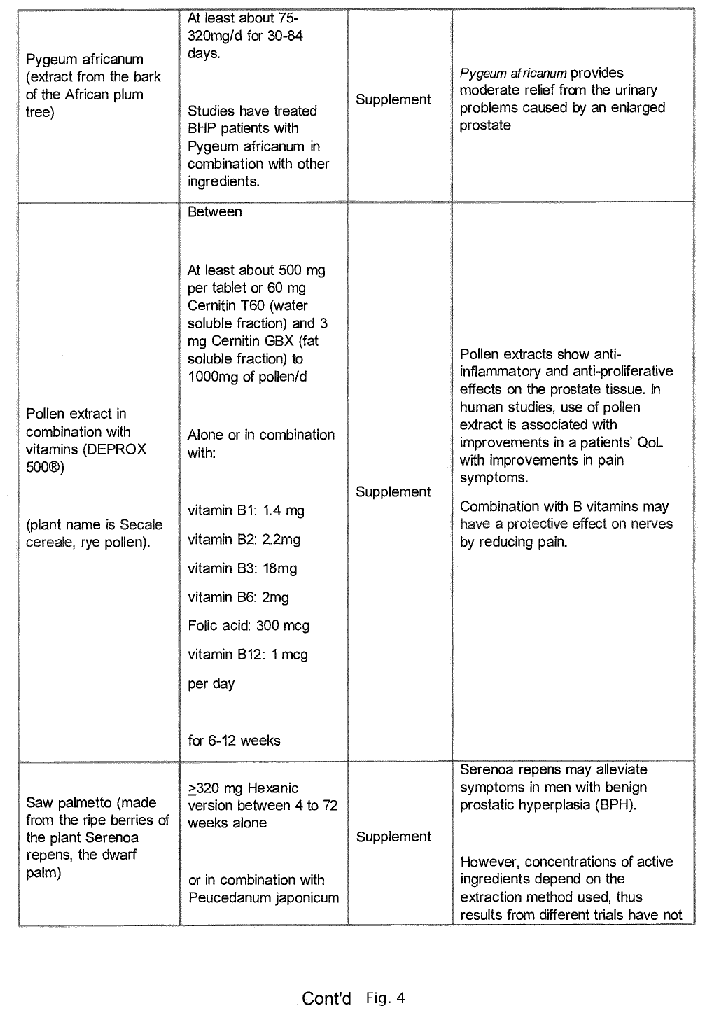

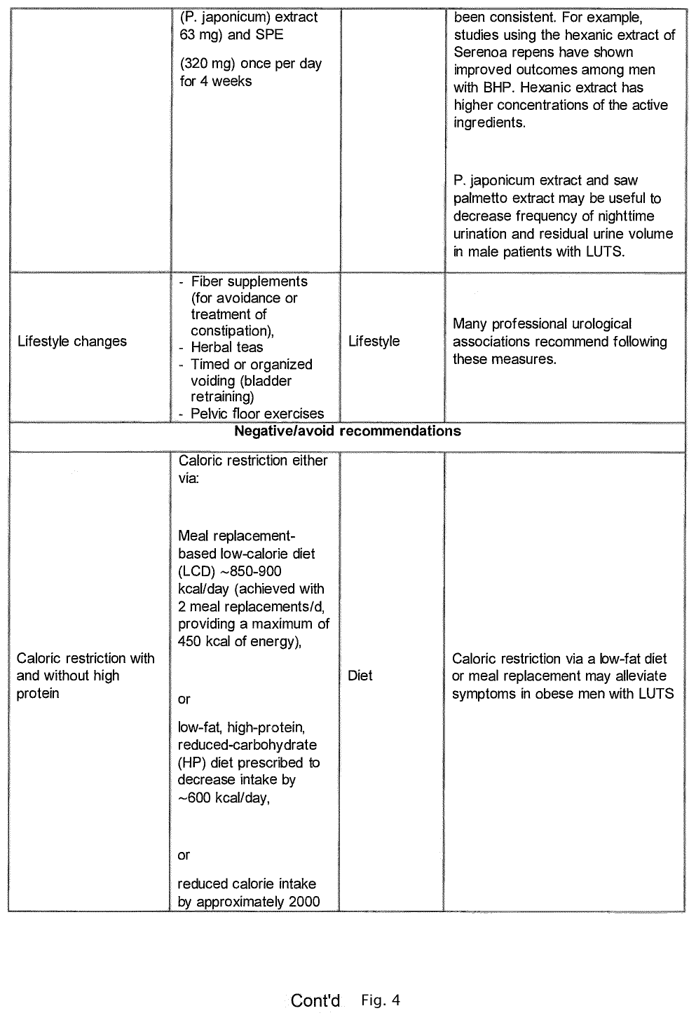

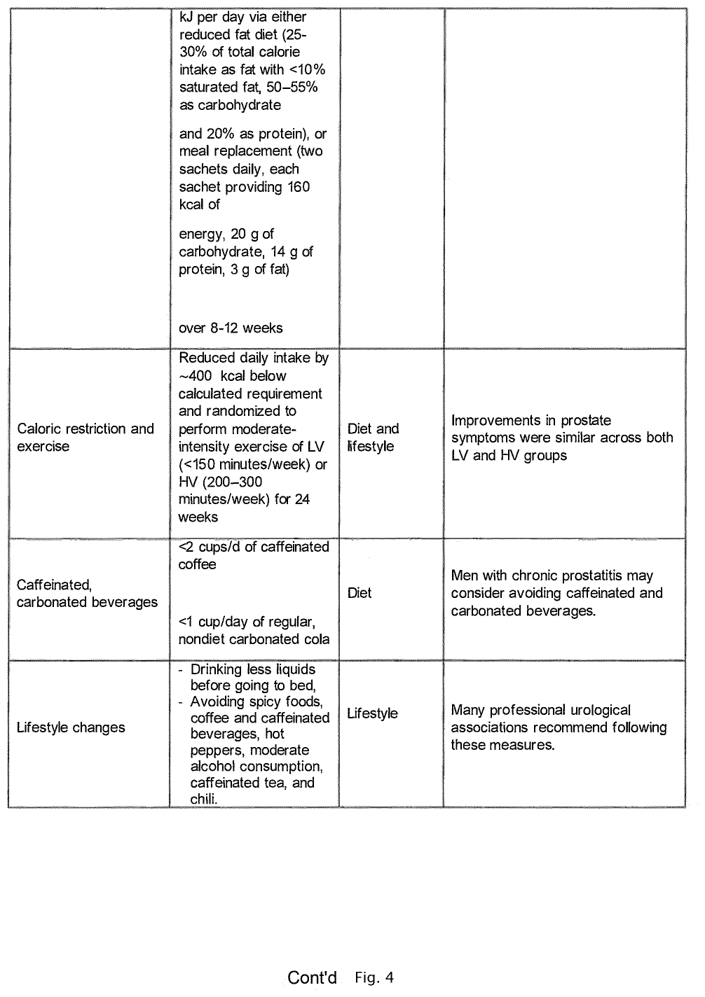

FIG. 4 illustrates an example plurality of dietary and lifestyle recommendations according to an exemplary embodiment of the present disclosure. This dietary recommendation example 400 details specific recommendations that may be presented to a user after a plurality of fertility enhancing recommendations 140 have been determined by the recommendation system 104. Specifically, example 400 details the fertility enhancing recommendations 140 as determined for a user that has a specific fertility-related medical condition 218. Specifically, example 400 represents the fertility enhancing recommendations as determined for a user with prostate conditions.

Other recommendations 140 may be to simply avoid, or increase, consumption of a particular food item. Similarly, the recommendations 140 may include recommendations to consume certain substances moderately or to prefer one substance over another. Though there are many different types of recommendations 140 found within the example 400, one should appreciate that any type of qualitative or quantitative recommendation may be made regarding these food items and nutrients.

Furthermore, the recommendation system 400 may generate fertility enhancing recommendations 140 that include lifestyle changes, such as altering activity level, increasing the number of hours of rest per night, taking action to reduce stress, or similar lifestyle-affecting actions. For example, a high level of stress may negatively affect a user's fertility. Such stress may derive from the relationship between the partners actively trying to conceive. Some example fertility enhancing recommendations 140 may include suggestions for methods by which the couple may decrease tensions in the relationship in order to alleviate stress. In another example, the fertility enhancing recommendations 140 may include recommendations to increase the amount of time a user rests, including sleeping habit recommendations. These recommendations may range from general recommendations, such as instruction to get more sleep, to more detailed recommendations, including specific exercise routines, specific diets and recipes, or suggested dates for visits to a medical professional.

In addition, in another embodiment, the fertility enhancing recommendations 140 generated by the recommendation system 104 may include specific recommendations for a product. For example, the recommendation system 104 may access a database containing information on a variety of supplements in the market. Then, based on its own analysis or through the use of third party research, the recommendation system 104 may analyze the different options for a specific supplement, such as Vitamin D, to determine that a specific supplement from a first brand, Brand A, is the most beneficial supplement as compared to other Vitamin D supplements available from a second, third, and fourth brand. Such an analysis may be conducted based on the quality of the supplements, the cost of the supplements, known side effects, method of manufacture, or any other factors that may distinguish the supplement provided by one brand from a supplement provided by another brand. The recommendation system 104 may provide similar recommendations as related to food items, such as a particular type or brand of apple, and any other category of product which may require the user to select one of multiple available options.

### Supplements

In some embodiments of the invention, the recommendation system recommends specific dietary supplements for individuals with prostate conditions who would like to improve their fertility.

In preferred embodiments of the invention, the dietary supplements are selected from the group of:

### Fermented Whey Protein

Whey protein is typically fermented by bacteria and processed into a powder to form a supplement.

### Vitamin D

Vitamin D is involved in the regulation of the minerals, calcium and phosphorus found in the body. It also plays an important role in maintaining proper bone structure. Typical sources of Vitamin D are from sunlight.

### Selenium

Selenium is a mineral which has antioxidant properties.

### Silymarin

Silymarin is a flavonolignans extracted from the milk thistle Silybum marianum (L.) gaernt. Silymarin has been shown to possess various pharmacological properties like hepato-protective, antioxidant, anti-inflammatory, anticancer, and cardio-protective activities.

Silymarin may be administered alone or in combination with selenium.

### Saw Palmetto

Saw palmetto is a supplement made from the fruit of the Serenoa repens tree used to treat enlarged prostate, improve urinary function, and enhance hair growth. Some also use the supplement to boost libido and fertility and reduce inflammation.

It may be administered alone or in combination with Peucedanum japonicum extract.

Peucedanum japonicum is also known as coastal hog fennel and is a species of Peucedanum, a genus rich in medicinal species belonging to the parsley family, Apiaceae.

### Cranberry

Cranberries contain compounds called proanthocyanidins, which prevent E. coli bacteria from attaching to the lining of your urethra and bladder thus helping to prevent urinary tract infections. Cranberry fruit supplements or cranberry juice may be taken to have this benefit.

### Carotenoids

Carotenoids are fat-soluble plant pigments which have anti-oxidant properties. Some of the carotenoids are converted into Vitamin A which is essential to vision and normal growth and development. The most common carotenoids in the diet are α-carotene, β-carotene, β-cryptoxanthin, lutein, zeaxanthin, and lycopene.

Lycopene may be found in foods such as tomatoes, asparagus and parsley. However, lycopene dietary supplements in oil may be more efficiently absorbed than lycopene from food.

β-carotene is converted into vitamin A, an essential vitamin and may be found in food such as onions, carrots, peas, spinach and squash. It may also be administered as a supplement.

### Curcumin

Curcumin is produced by Curcuma longa plants and typically in a bright yellow powder. It is the principal curcuminoid of turmeric (Curcuma longa), a member of the ginger family, Zingiberaceae. It has potent anti-inflammatory and antioxidant properties.

### Soy Isoflavones

In soybeans, isoflavones are present as glycosides, i.e., bound to a sugar molecule. They exhibit antioxidant, anticancer, antimicrobial, and anti-inflammatory properties.

### Flavonoids

Flavonoids are a class of polyphenolic plant and fungus secondary metabolites. Quercetin is a flavonoid that is found in many plants and foods, such as red wine, onions, green tea, apples, berries, Ginkgo biloba, St. John's wort, American elder, and others. Buckwheat tea has a large amount of quercetin. Quercetin has antioxidant and anti-inflammatory effects.

### Flaxseed Lignan Extract

Flax is particularly high in the lignans secoisolariciresinol diglucoside (SDG), SECO, and matairesinol. The main lignan in flax is secoisolariciresinol diglucoside (SDG). When ingested, micro-organisms in the colon convert SDG into enterodiol and enterolactone, the so-called mammalian lignans. These are the body's own active forms of lignans.

### Beta-Sitosterol Extract

Beta-sitosterol is a plant substance similar to cholesterol. Beta-sitosterol extract is typically extracted from Hypoxis rooperi, the South African star grass. It helps to reduce cholesterol levels by limiting the amount of cholesterol that is able to enter the body. It can also bind to the prostate to help reduce swelling and inflammation.

### Urtica dioica Extract

Urtica dioica, often known as common nettle, stinging nettle is a herbaceous perennial flowering plant in the family Urticaceae. It has been used for urination problems related to an enlarged prostate (benign prostatic hyperplasia [BPH]). These problems include nighttime urination, too frequent urination, painful urination, inability to urinate, and irritable bladder.

### Pygeum africanum

Pyceum africanum extract is from the African prune tree is used for treating symptoms of enlarged prostate (benign prostatic hyperplasia, BPH) and prostate cancer. It is also used for pain caused by inflammation, kidney disease, urinary problems, malaria, stomachache, fever, and to increase sexual desire.

### Secale cereale (Rye Pollen Extract)

Secale cereale is a rye grass pollen extract which is used to make supplements.

It may be administered alone or in combination with other vitamins such as: vitamin B1, vitamin B2, vitamin B3, vitamin B6, Folic acid, and vitamin B12.

In a preferred embodiment of the invention, the recommendation for supplements is to be administered as separate supplements or in combination selected from the group consisting of:

(i) Fermented whey protein in the amount of at least about 200 g/day for at least 4 weeks;

(ii) Vitamin D in the amount of at least about 15 ug/day;

(iii) Selenium in the amount of at least about 240 ug/day for at least 6 months;

(iv) Silymarin in the amount of at least 570 mg/day for at least 6 months;

(v) Saw palmetto in the amount of at least 320 mg/day for at least 72 weeks;

(vi) Cranberry powder in the amount of 250 to 1500 mg/day;

(vii) Carotenoids selected from: lycopene in the amount of at least 15 mg/day for at least 6 months and beta-carotene in the amount of at least 15 mg/day for at least 6 months;

(viii) Curcumin in the amount of 200 to 1000 mg/day for at least 5.5 months;

(ix) Soy isoflavones in the amount of 40 mg/day for at least 12 months;

(x) Quercetin flavonoid in the amount of 500 -1000 mg/day for at least 4 weeks;

(xi) Flaxseed lignan extract in the amount of 300-600 mg/day;

(xii) Beta-sitosterols in the amount of 60-195 mg/day for at least 26 weeks;

(xiii) Urica dioica extract in the amount of 120-600 mg/day for at least 6 months;

(xiv) Pygeum africanum in the amount of 75-320 mg/day for at least 84 days; and

(xv) Secale cereal, rye pollen extract in the amount of 500-1000 mg/day;

In one embodiment, the recommendation for supplements is to be administered in the combination of selenium and silymarin for at least 6 months.

In another embodiment, the recommendation for supplements is to be administered in the combination of Saw palmetto and Peucedanum japonicum extract for at least 4 weeks.

In a further embodiment, the recommendation for supplements is to be administered in the combination of Secale cereale with vitamins selected from the group consisting of: vitamin B1, vitamin B2, vitamin B3, vitamin B6, folic acid, and vitamin B12.

In several embodiments of the invention, the recommendation system recommends specific supplements combined with food items for individuals with prostate conditions who would like to improve their fertility.

### Food Items

In some embodiments of the invention, the recommendation system recommends specific food items for individuals with prostate conditions who would like to improve their fertility.

In some embodiments of the invention, the recommendation system recommends specific food items for individuals with prostate conditions who would like to improve their fertility selected from the group:

### Onions

Onions contains antioxidants and compounds that fight inflammation, decrease triglycerides and reduce cholesterol levels which may lower heart disease risk. Their potent anti-inflammatory properties may also help reduce high blood pressure and protect against blood clots. Allium species, particularly the onion (Allium cepa) and garlic (A. sativum). The recommended amount of onion extract is at least about 30 mg/day.

### Garlic

Garlic is an excellent source of minerals and vitamins that are essential for optimum health. The bulbs are one of the richest sources of potassium, iron, calcium, magnesium, manganese, zinc, and selenium. Selenium is a heart-healthy mineral and is an essential cofactor for antioxidant enzymes within the body.

### Citrus Juice

Citrus juice from oranges, lemons, limes, and grapefruits are all high in vitamin C, which may help to protect the prostate gland.

### Vegetable and Fruits

Adequate fruit and vegetable consumption may alleviate lower urinary tract symptoms (including overall, storage, and voiding symptoms) including benign prostatic hyperplasia.

Per 2000 kcal/day diet one should consume at least 700 g total vegetables and fruits. Of the at least 400 g vegetables at least 100 g are dark, leafy vegetables. Of the at least 300 g fruit, at least 20 g are tomatoes.

### Pumpkin Seeds or Pumpkin Seed Extract

Pumpkin seeds are also known as Cucurbita pepo. Whole pumpkin seeds or pumpkin seed extract may be used. Pumpkin seed or pumpkin seed extract in oil can help prevent the multiplication of overstimulated prostate cells, levelling hormones.

In one embodiment of the invention, pumpkin seed extract may be combined to be administered together with soy isoflavonoids and cranberry.

In one embodiment of the invention, pumpkin seed extract may be combined to be administered together with Saw palmetto, Urtica dioica extract, Lemon bioflavonoid extract and β-carotene.

In a preferred embodiment of the invention, the recommendation for food is to be selected from the group consisting of:

(i) onions in the amount of at least 4 portions each week for 4 weeks;

(ii) garlic in the amount of at least 4 portions each week for 4 weeks;

(iii) citrus juice in the amount of at least 240 ml per day;

(iv) vegetables and fruits in the amount of at least about 700 g per 2000 kcal diet: at least 400 g vegetables of which at least 100 g are dark, leafy vegetables and at least 300 g fruit of which at least 20 g are tomatoes; and

(v) pumpkin seeds in the amount of at least about 5 g/day or pumpkin seed extract in the amount of at least about 500 mg/day.

### Specific Diet and Lifestyle Changes

In some embodiments of the invention, the recommendation system recommends specific diets or lifestyle changes for individuals with prostate conditions who would like to improve their fertility.

In one embodiment of the invention, the recommendation system recommends fiber supplements for avoidance or treatment of constipation.

In one embodiment of the invention, the recommendation system recommends consumption of herbal teas to ensure sufficient daily hydration.

In one embodiment of the invention, the recommendation system recommends timed or organized voiding of bladder to avoid risk of urinary tract infections.

In one embodiment of the invention, the recommendation system recommends pelvic floor muscle exercises.

In one embodiment of the invention, the recommendation system recommends caloric restriction with and without high protein in obese men with a BMI over 25 kg/m2 to order to lose weight.

In one embodiment of the invention, the recommendation system recommends reducing alcoholic beverages and carbonated beverages to less than one per day.

In one embodiment of the invention, the recommendation system recommends reducing caffeinated beverages to less than two per day.

In one embodiment of the invention, the recommendation system recommends avoiding spicy foods and reducing hot peppers and chili.

In several embodiments of the invention, the dietary recommendations of the present invention may contribute to improving prostate-related symptoms inflammation of the prostate or lower urinary tract symptoms (LUTS).

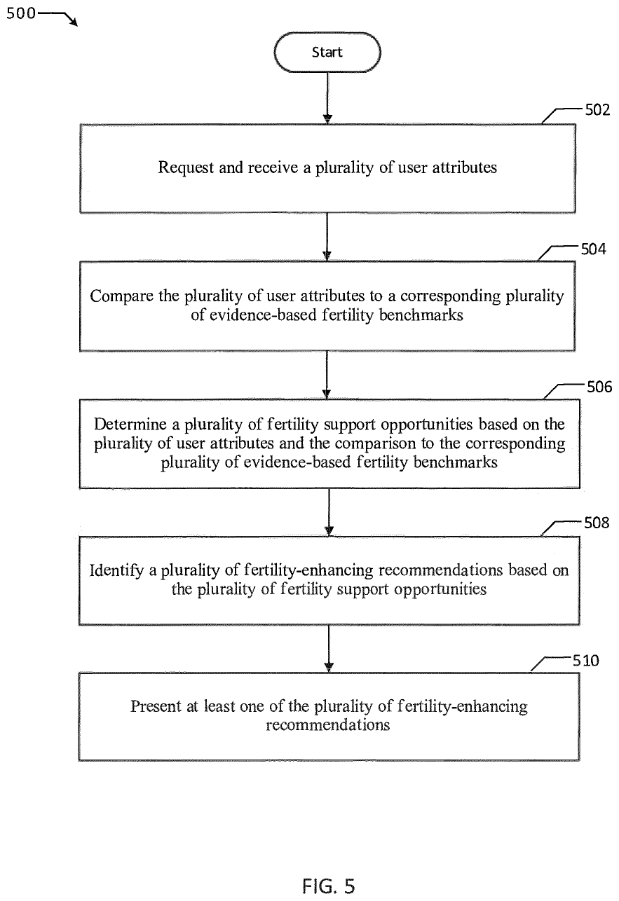

FIG. 5 illustrates an example embodiment of a method 500 of the presently disclosed method, as was discussed above in relation to the system 100. The method 500 may be implemented in a system, such as the system 100, or on a CPU. For example, the method may be implemented by one or more of the attribute receiving unit 108, the attribute analysis unit 114, the attribute storing unit 116, the attribute comparison unit 110, the evidence-based diet and lifestyle recommendation engine 112, or the user device 102. The method 500 may also be implemented by a set of instructions stored on a computer readable medium that, when executed by a processor, cause the computer system to perform the method. For example, all or part of the method 500 may be implemented by the CPU 120 and memory 118. Although the examples below are described with reference to the flowchart illustrated in FIG. 5, many other methods of performing the acts associated with FIG. 5 may be used. For example, the order of some of the blocks may be changed, certain blocks may be combined with other blocks, one or more of the blocks may be repeated, and some of the blocks described may be optional.

Block 502 can include requesting and receiving a plurality of user attributes 122. For example, a display 106 may present an attribute questionnaire 124 to solicit answers 126, to which the user device 102 provides the answers 126 to then be selected as user attributes 122. In block 504, comparisons of the plurality of user attributes 122 to a corresponding plurality of evidence-based fertility benchmarks 128 may occur. Based on these comparisons, at block 506, a plurality of fertility support opportunities 138 can be determined based on the plurality of user attributes 122 and the comparison to the corresponding plurality of evidence-based fertility benchmarks 128. At block 508, an embodiment of method 500 may identify a plurality of fertility enhancing recommendations 140 based on the plurality of fertility support opportunities 138. For example, the evidence-based diet and lifestyle recommendation engine 112 may comprise a cloud-based system trained to interpret fertility support opportunities to provide recommendations 140. Lastly, at block 510, at least one of the plurality of fertility enhancing recommendations 140 can be presented.

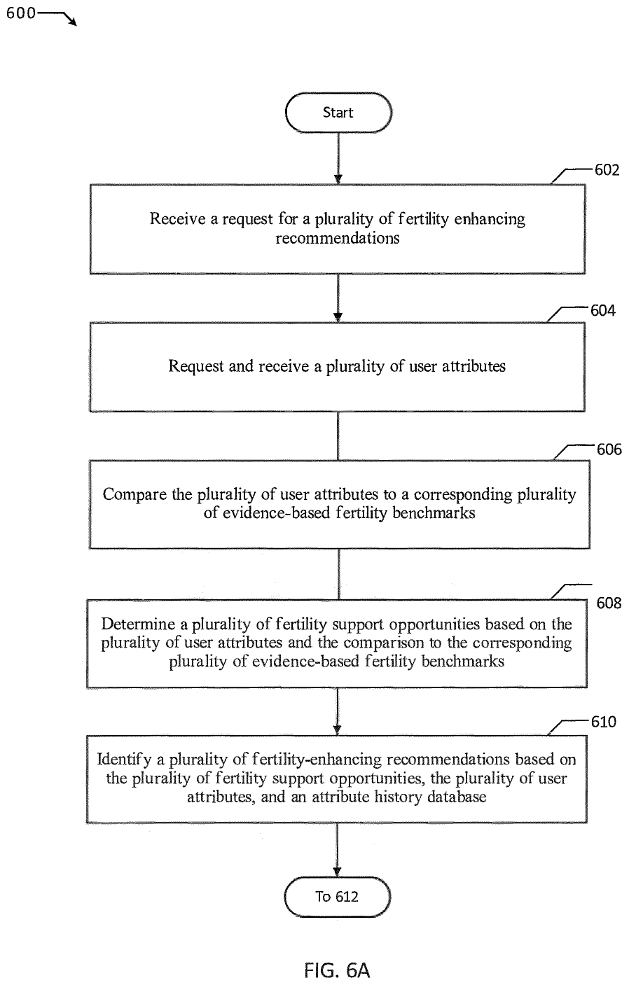

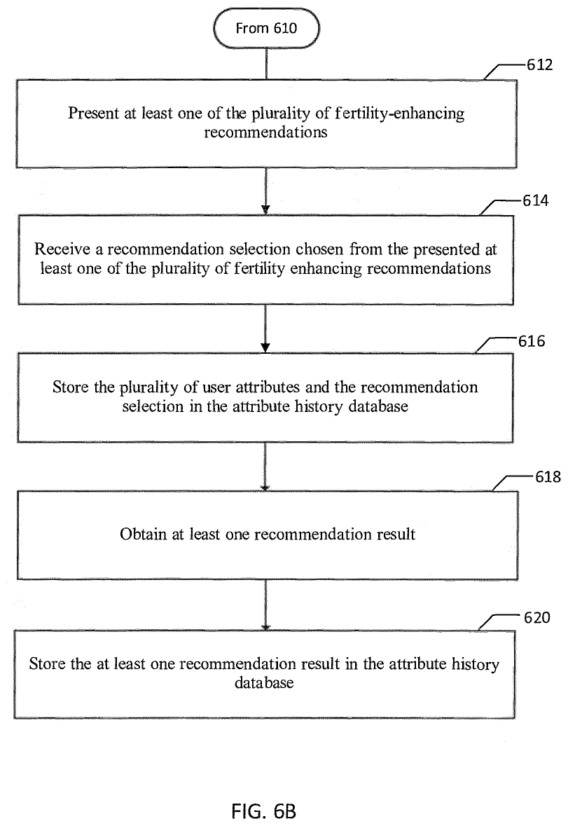

FIGS. 6A and 6B disclose an exemplary embodiment of a method 600 of the presently disclosed method. The method 600 may be implemented in a system, such as the system 100, or on a CPU. For example, the method may be implemented by one or more of the attribute receiving unit 108, the attribute analysis unit 114, the attribute storing unit 116, the attribute comparison unit 110, the evidence-based diet and lifestyle recommendation engine 112, or the user device 102. The method 600 may also be implemented by a set of instructions stored on a computer readable medium that, when executed by a processor, cause the computer system to perform the method. For example, all or part of the method 600 may be implemented by the CPU 120 and memory 118. Although the examples below are described with reference to the flowchart illustrated in FIG. 6, many other methods of performing the acts associated with FIG. 6 may be used. For example, the order of some of the blocks may be changed, certain blocks may be combined with other blocks, one or more of the blocks may be repeated, and some of the blocks described may be optional.

Block 602 may include receiving a request for a plurality of fertility enhancing recommendations 140. For example, a user may submit a request for the fertility enhancing recommendations 140 through any number of methods, including: opening an application on the user device 102, making a formal request through an application on the user device 102, submitting a request for periodic fertility enhancing recommendations 140 through the user device 102, signing into an online account through a web browser, making a formal request through a web browser, or submitting a request for periodic fertility enhancing recommendations 140 through the web browser.

At block 604, the recommendation system 104 may request and receive a plurality of user attributes 122. For example, the recommendation system 104 may present an attribute questionnaire 124 to the user. This attribute questionnaire 124 may be a standard questionnaire or a questionnaire that is customized based on known preliminary attributes, or answers to prior questions. In another example, the recommendation system 104 may request the plurality of user attributes 122 by providing a list of available home test kits, that a user may use at home. Then, after the test has been performed, the recommendation system 104 may receive the results from the test and, based on these results, determine the user attributes 122 related to such a test. For example, the home test kit may be an application to track the timing of the ovulation cycle of the user to determine the best dates for conception which may be monitored by a further application on an additional user device.

In another example, at block 604, the recommendation system 104 may provide a self-assessment tool. Similar to the prior example, the user will may make use of this self-assessment tool, submitting the results to the recommendation system 104. Again, based on the received results, the recommendation system 104 may determine the user attributes 122 based on the test. In yet another example, the recommendation system 104 may request the user have a standardized health test performed by a medical professional. In this example, the results of this performed health test may be submitted to the recommendation system 104, which thereby determines the user attributes 122 based on the results. Though some specific examples as to external tests have been given, these examples are non-limiting as the recommendation system 104 may be configured to receive results of any external or third party test in order to determine the corresponding user attributes 122.

In Block 606, the recommendation system 104 may be configured to compare the plurality of user attributes 122 to a corresponding plurality of evidence-based fertility benchmarks 128. For example, these evidence-based fertility benchmarks 128 may include standardized benchmarks, as in benchmarks that are given to all, regardless of individual variances. In another example, these benchmarks 128 may be customized based on a particular user's history or goals. For example, if a healthy user is trying to improve his or her fertility and the current user attributes 122 exceeds all standard evidence-based fertility benchmarks 128, the recommendation system 104 may be configured to determine a customized fertility benchmark set 132 for which the particular user should aim. In contrast, in another example, a different user that is far below a standard evidence-based fertility benchmark 128 may be compared to a different, lower benchmark value as a manner of inspiring progress and providing milestones.

The example method, at Block 608, may be configured to determine a plurality of fertility support opportunities 138 based on the plurality of user attributes 122 and the comparison to the corresponding plurality of evidence-based fertility benchmarks 128. For example, the recommendation system 104 may determine that a user attribute 122 corresponds to an above optimal stress level. Based on this comparison, the recommendation system 104 may determine a fertility support opportunity 138 to reduce stress. In another example, the recommendation system 104 may determine that the user has not yet seen a medical professional, and, as such, determine a fertility support opportunity 138 to visit a medical professional.

At Block 610, the recommendation system 104 may identify a plurality of fertility-enhancing recommendations 140 based on at least the plurality of fertility support opportunities 138. For example, the recommendation system may determine a plurality of similar prior cases by analyzing the attribute history database 148, identifying similarities between the user attributes 122 received and the plurality of prior user attributes within the attribute history database 148. For example, the recommendation system 104 may identify that the user attributes 122 detail a user with an above average BMI and other similarities that correspond to a particular group of past users and therefore the cases of those member of that particular group of past users are determined as similar prior cases.

Furthermore, in this example, the recommendation system 104 may determine a plurality of prior case results based on the plurality of similar prior cases. As detailed previously, the attribute history database 148 may comprise corresponding recommendations associated with prior user attributes, and the effectiveness of these corresponding recommendations. As such, the recommendation system 104 may analyze the corresponding recommendations and their effectiveness as associated with the particular group of past users to determine a plurality of prior case results.

Furthermore, in this example, the recommendation system 104 may determine successful recommendations and a plurality of unsuccessful recommendations based on a plurality of prior case results. For example, the recommendation system 104 may have recommended the users in that particular group of past users increase exercise levels in some cases and decrease food consumption in other cases. Based on prior case results as determined based on the attribute history database 148, the recommendation system 104 may determine that the recommendations for decreasing food consumption were not very successful, yet increasing exercise levels proved to be very successful and, as such, determines that increasing exercise levels is a successful recommendation whereas decreasing food consumption is an unsuccessful recommendation. By conducting analysis of these prior user attributes, the recommendation selection and the effectiveness of corresponding recommendations, the recommendation system 104 may identify trends associated with different subset patient populations, thereby creating and validating a plurality of lifestyle interventions. These examples of successful and unsuccessful recommendations are nonlimiting, as different groups may experience different levels of success to the same recommendations.

In addition, the recommendation system 104 may be configured to determine a plurality of fertility enhancing recommendations based on the plurality of successful recommendations and the plurality of unsuccessful recommendations. For example, the recommendation system 104 may be configured to only recommend the plurality of successful recommendations. In another example, the recommendation system 104 may still recommend any of the unsuccessful recommendations. The recommendation system 104 may make these recommendations based on any number of reasons, including a slight difference in the user attributes 122 as compared to the prior user attributes, a lack of insufficient data to support a true unsuccessful recommendation, or data supporting that, although unsuccessful, the recommendation is popular and often followed through by users. In another example, the recommendation system 104 may recommend less than all of the plurality of successful recommendations. In an example, the decision for selecting which of the plurality of recommendations to present generate may be performed by an AI.

In another example, successful recommendations may be based on guidelines associated with particular medical conditions, such as a user with prostate issues. In that case, these guidelines would be determined as successful recommendations.

At Block 612, the recommendation system may present at least one of the plurality of fertility-enhancing recommendations 140. At Block 614, the recommendation system 104 may receive a recommendation selection chosen from the presented at least one of the plurality of fertility enhancing recommendations 140. For example, a user may be presented with three fertility-enhancing recommendations 140, to follow avoid a spicy diet; to increase consumption of fruits and vegetables; and to reduce alcohol consumption to less than one drink per day. The user may select one, two, or all three of these options. As such, the recommendation system 104 receives, from the user device 102, these two selected recommendations as the recommendation selection. In another example, the user may not select any of the presented recommendations, at which point the recommendation system 104 may generate and present a different plurality of fertility enhancing recommendations 140.

In another example, after the user reviews the presented fertility enhancing recommendations 140, the user may submit a request to contact a fertility coach. For example, the user may be undecided as to how to implement the recommendations or may simply have a question for which the user seeks an answer. In some instances, the recommendation system 104 may determine that the question for which the user seeks an answer may be adequately answered by a virtual coach, and thereby provides access to and interaction with said virtual coach. In other instances, the recommendation system 104 may determine that the question will be best handled by a personal coach, a living individual, and thereby provides access to and interaction with said personal coach.

At Block 616, the recommendation system 104 may store the plurality of user attributes 122 and the recommendation selection in the attribute history database 148. For example, the recommendation system 104 may store all user attributes 122 received on a first day, along with the recommendation selection received on that same first day. These user attributes 122 and recommendation selection may then be accessed by the recommendation system 104 in the future when analyzing the attribute history database 148.

The recommendation system 104 may obtain at least one recommendation result at Block 618. In an example, the user may submit a recommendation result through the user device 102. This result may include a qualitative or quantitative rating as selected by the user. In another example, the recommendation system 104 may receive a future plurality of user attributes 122 and, at that time, compare the received future user attributes with the previously received user attributes, now prior user attributes, within the attribute history database 148. Based on this comparison, the recommendation system 104 may determine a recommendation result, such as decreased or increased BMI. After obtaining this recommendation result, the recommendation system 104 may store the at least one recommendation result in the attribute history database 148, corresponding to the prior recommendation selection. This recommendation system 104 may then await another request for fertility enhancing recommendations 140, and at that time, perform the method 600 again at Block 602.

Such an example method as disclosed in FIGS. 6A and 6B allow for the continuous, customized, integrated recommendation system 104 to endlessly improve upon recommendations as the attribute history database 148 grows in size. Through this growth, the recommendation system 104, and in some embodiments, the evidence-based diet and lifestyle recommendation engine 112, will have an ever-expanding set of data from which it can derive fertility enhancing recommendations 140, with increasing particularity regarding what users receive which recommendations.

In another aspect, a method of treatment may comprise using any of the above described systems or methods to generate any one or more of the fertility enhancing recommendations 140, diet and lifestyle recommendations, or specific supplementation recommendations. Furthermore, the method of treatment may comprise administering a treatment based on at least the any of one or more of the fertility enhancing recommendations 140, diet and lifestyle recommendations, or specific supplementation recommendations to a user. For example, when the recommendation system 104 determines a fertility enhancing recommendation 140 that comprises increasing a user's selenium to 240 ug/day by way of a supplement, an example method of treatment may comprise administering a treatment comprising a selenium supplement of 240 ug to the user each day.

All of the disclosed methods and procedures described in this disclosure can be implemented using one or more computer programs or components. These components may be provided as a series of computer instructions on any conventional computer readable medium or machine-readable medium, including volatile and non-volatile memory, such as RAM, ROM, flash memory, magnetic or optical disks, optical memory, or other storage media. The instructions may be provided as software or firmware, and may be implemented in whole or in part in hardware components such as ASICs, FPGAs, DSPs, or any other similar devices. The instructions may be configured to be executed by one or more processors, which when executing the series of computer instructions, performs or facilitates the performance of all or part of the disclosed methods and procedures.

It should be understood that various changes and modifications to the examples described here will be apparent to those skilled in the art. Such changes and modifications can be made without departing from the spirit and scope of the present subject matter and without diminishing its intended advantages. It is therefore intended that such changes and modifications be covered by the appended claims.

### EXAMPLES

### Example 1: Dietary Recommendations for Individuals with Prostate Conditions

The following are dietary recommendations or supplements for individual with prostate conditions.

| | | | | |
|---|---|---|---|---|
| Diet and | Dietary | Supplement or | | |
| lifestyle components | recommendation | Diet or lifestyle | Tips for the consumer | Ref |

Positive recommendations

| | | | | |
|---|---|---|---|---|
| Fermented | At least about 200 g | Supplement | Fermented whey protein may | [1] |
| whey protein | per day for 4 weeks | | improve urinary function and | |
| | | | prostate-related characteristics in | |
| | | | men with lower urinary tract | |
| | | | symptoms. | |
| Onion and | High use of garlic | Diet | Men who eat significant amounts | [2] |
| garlic | and onions, 4 or | | onions and garlic per week have | |
| | more portions per | | less often benign prostatic | |
| | week | | hyperplasia (BPH). Particularly | |
| | | | the combination of onion and | |
| | | | garlic consumption seems to be | |
| | | | beneficial. | |
| Citrus juice, | At least about >240 | Diet | Citrus juice and herbal teas could | [3, 4] |
| herbal teas | ml Citrus juice per | | potentially alleviate progression of | |
| | day | | LUTS symptoms in men with | |
| | At least about 240 ml | | chronic prostatitis. | |
| | per day of herbal tea; | | | |
| Vitamin D | At least about 15 μg | Supplement | Adequate vitamin D intake to | [5, 6] |
| | per day* | | avoid deficiency may alleviate | |
| | | | symptoms of overactive bladder | |
| | | | in men with lower urinary tract | |
| | | | symptoms. | |
| Fruit and | At least 350 g per | Diet | Adequate fruit and vegetable | [7] |
| vegetables | 1000 kcal/day total | | consumption may alleviate lower | |
| | fruits and vegetables | | urinary tract symptoms (including | |
| | At least 150 g per | | overall, storage, and voiding | |
| | 1000 kcal/day total | | symptoms) including benign | |
| | vegetables | | prostatic hyperplasia. | |
| | At least 50 | | Per 2000 kcal/day diet: | |
| | g/1000 kcal/day dark | | At least 700 g total fruit and | |
| | and leafy vegetable | | vegetables | |
| | At least 10 g/1000 | | At least 300 g vegetables of | |
| | kcal/day tomatoes | | which | |
| | At least 150 g/1000 | | at least 100 g are dark leafy | |
| | kcal/day total fruit | | vegetables | |
| | | | At least 300 g fruit of which at | |
| | | | least 20 g tomatoes | |
| Selenium and | At least about 240 μg | Supplement | Selenium and silymarin may be | [8, 9] |
| silymarin | selenium plus at | | beneficial for men with lower | |
| | least about 570 mg | | urinary tract symptoms. | |
| | silymarin for 6 | | | |
| | months | | | |
| | or in combination as | | | |
| | follows: | | | |
| | 320 mg of Saw | | | |
| | palmetto from | | | |
| | Serenoa repens | | | |
| | plant (supercritical | | | |
| | CO2 lipidic extract | | | |
| | containing 85% of | | | |
| | fatty acids sterols, | | | |
| | selenium (50 ug) and | | | |
| | lycopene (5 mg)) | | | |
| Cranberry | Cranberry fruit | Supplement | Cranberry extracts may | [10, 11] |
| | powder 250 mg- | | ameliorate symptoms of LUTS in | |
| | 500 mg for 6 months | | males with benign prostatic | |
| | 1500 mg cranberry | | hyperplasia (BPH). | |
| | fruit powder | | | |
| | (Vaccinium | | | |
| | macrocarpon) for 6 | | | |
| | months | | | |
| Carotenoids | At least about 15 | Supplement | Slow the progression of benign | [12-14] |
| (lycopene, β- | mg/day of lycopene | | prostate hyperplasia | |
| carotene) | for 6 months | | | |
| | Or in combination as | | | |
| | follows: | | | |
| | herbal formulation | | | |
| | containing C. pepo | | | |
| | seed oil (160 mg), | | | |
| | E. parviflorum | | | |
| | extract | | | |
| | (equivalent to 500 | | | |
| | mg dry herb), | | | |
| | lycopene (2.1 mg), | | | |
| | Prunus Africana | | | |
| | (equivalent to 15 g | | | |
| | dry stem, | | | |
| | standardized | | | |
| | to b-sitosterol) and | | | |
| | S. repens (equivalent | | | |
| | to 660 mg of dry | | | |
| | leaf per capsule) | | | |
| | for 3 months | | | |
| Curcumin | At least about 2 × | Supplement | Men with benign prostatic | [15, 16] |
| | 100 mg/day curcumin | | hyperplasia may benefit from | |
| | alone | | curcumin in addition to the | |
| | Or in combination as | | standard treatment. | |
| | follows: | | Improvements observed in | |
| | 135 mg pine bark | | nocturia, urgency, hesitancy, | |
| | (pinus ssp. with a | | dysuria, poor and/or intermittent | |
| | minimal beta- | | stream, feeling of incomplete | |
| | sitosterol content of | | bladder emptying and post- | |
| | 94.5 mg), 105 mg | | micturition dribble. This may be | |
| | turmeric (with a | | due to the anti-inflammatory and | |
| | curcuminoid content | | antioxidant properties of | |
| | of 99.75 mg) and | | curcumin. | |
| | 21 mg of pine bark | | | |
| | (pinus massoniana | | | |
| | with an oligomeric | | | |
| | proantocianidine | | | |
| | content of 19.95 mg) | | | |
| | per day | | | |
| | For 3 to 5.5 months | | | |
| Soy | At least about 40 mg | Supplement | Soy isoflavones may improve | [17] |
| isoflavones | for 12 months | | symptoms in men with benign | |
| (Soylife40) | | | prostatic hyperplasia (BPH). | |
| Flavonoids | At least about | Supplement | Flavonoids may have clinical | [18-21] |
| | Quercetin (500 mg | | benefits in in chronic prostatitis | |
| | twice a day) for 4 | | patients by improving symptoms | |
| | weeks | | | |
| Flaxseed | At least about 300 to | Supplement | Lignan from flaxseed may | [22] |
| lignan | 600 mg/day | | ameliorate prostate-related | |
| extract | (flaxseed lignan | | problems in men with benign | |
| | extract containing | | prostatic hyperplasia (BPH). | |
| | 33% | | | |
| | secoisolariciresinol | | | |
| | diglucoside (SDG)) | | | |
| Beta- | At least about 60-195 | Supplement | B-Sitosterols improved urinary | [23, 24] |
| sitosterols | mg/day for 4 to 26 | | symptom scores and flow | this |
| (extract made | weeks | | measures. | reference |
| from Hypoxis | Studies utilized | | Beta-sitosterols are found in saw | is a |
| rooperi, the | purified extracts from | | palmetto, rye grass pollen, | meta- |
| South African | a variety of plant | | pygeum, and stinging nettles. | analysis |
| star grass) | species. | | | of |
| | | | | several |
| | | | | studies |
| Urtica dioica | At least about 120 | Supplement | Urtica dioica is composed of | [25, 26] |
| (stinging | mg/dose 3 times | | phytosterols, triterpenic acids, | |
| nettle) | daily (total of 360 | | lignans, polysaccharides and | |
| | mg/day) for 6 months | | simple phenol compounds. It may | |
| | or at least about | | have inhibitory effects on | |
| | 300 mg, 2 times a | | inflammatory | |
| | day for 8 weeks | | mediators by prevent binding of | |
| | | | androgens to sex hormone | |
| | | | binding globulin. For example, | |
| | | | rats studies have shown that | |
| | | | administration of urtica dioica | |
| | | | leads to a significant reduction in | |
| | | | prostate volume by a decrease in | |
| | | | both the percentage of folded | |
| | | | alveoli in the ventral lobe and | |
| | | | their diameters. | |
| Pumpkin | At least about 5 g of | Diet | Pumpkin seeds may be beneficial | [27-31] |
| seeds (plant | Pumpkin seeds for | | in improving symptoms of benign | |
| name is | 12 months | | prostatic hyperplasia (BPH). | |
| Cucurbita | or | | Pumpkin seed may have an | |
| pepo) | one tablet daily of oil- | | antioxidant, antiandrogen, and | |
| | free hydroethanolic | | anti-inflammatory activity. | |
| | pumpkin seed extract | | | |
| | (at least about 500 | | | |
| | mg of the extract, | | | |
| | corresponding | | | |
| | to 350 mg native | | | |
| | extract and | | | |
| | equivalent to 10 g of | | | |
| | pumpkin seeds per | | | |
| | tablet) | | | |
| | for 12 weeks | | | |
| | or | | | |
| | in combination with | | | |
| | other ingredients. | | | |
| | Pumpkin seed | | | |
| | extract 550 mg, soy | | | |
| | germ isoflavonoids | | | |
| | 50 mg, and cranberry | | | |
| | 50 mg | | | |
| | Or as a blend: | | | |
| | Saw palmetto lipoidal | | | |
| | extract (mg.) 106 | | | |
| | Nettle root extract | | | |
| | (80 mg), Pumpkin | | | |
| | seed oil extract | | | |
| | (160 mg), | | | |
| | Lemon bioflavonoid | | | |
| | extract (33 mg), | | | |
| | Vitamin A, 100% as | | | |
| | β-carotene (190 IU). | | | |
| | Dose was 1 tablet 3 | | | |
| | times daily with | | | |
| | meals or 318 mg. | | | |
| | saw palmetto extract | | | |
| | daily. | | | |
| | Or as a blend: | | | |
| | pumpkin seed oil | | | |
| | (320 mg/day), saw | | | |
| | palmetto oil (320 | | | |
| | mg/day) or pumpkin | | | |
| | seed oil plus saw | | | |
| | palmetto oil (each | | | |
| | 320 mg/day) for 12 | | | |
| | months, with | | | |
| | improvement in | | | |
| | International prostate | | | |
| | symptom scores | | | |
| | already observed at | | | |
| | 3 months | | | |
| Pygeum | At least about 75- | Supplement | Pygeum africanum provides | [32] |
| africanum | 320 mg/d for 30-84 | | moderate relief from the urinary | this |
| (extract from | days. | | problems caused by an enlarged | reference |
| the bark of the | Studies have treated | | prostate | is a |
| African plum | BHP patients with | | | meta- |
| tree) | Pygeum africanum | | | analysis |
| | in combination | | | of |
| | with other | | | several |
| | ingredients. | | | studies |
| Pollen extract | Between | Supplement | Pollen extracts show anti- | [20, 21, |
| in combination | At least about 500 | | inflammatory and anti-proliferative | 33-36] |
| with vitamins | mg per tablet or 60 | | effects on the prostate tissue. In | |
| (DEPROX | mg (water soluble | | human studies, use of pollen | |
| 500 ®) | fraction) and 3 mg | | extract is associated with | |
| (plant name is | (fat soluble fraction) | | improvements in a patients' QoL | |
| Secale | to 1000 mg of | | with improvements in pain | |
| cereale, rye | pollen/day | | symptoms. | |
| pollen). | Alone or in | | Combination with B vitamins may | |
| | combination with: | | have a protective effect on nerves | |
| | vitamin B1: 1.4 mg | | by reducing pain. | |
| | vitamin B2: 2.2 mg | | | |
| | vitamin B3: 18 mg | | | |
| | vitamin B6: 2 mg | | | |
| | Folic acid: 300 mcg | | | |
| | vitamin B12: 1 mcg | | | |
| | per day | | | |
| | for 6-12 weeks | | | |
| Saw palmetto | ≥320 mg Hexanic | Supplement | Serenoa repens may alleviate | [12, |
| (made from | version between 4 to | | symptoms in men with benign | 36-41] |
| the ripe berries | 72 weeks alone | | prostatic hyperplasia (BPH). | |
| of the plant | or in combination | | However, concentrations of active | |
| Serenoa | with Peucedanum | | ingredients depend on the | |
| repens, the | japonicum | | extraction method used, thus | |
| dwarf palm) | (P. japonicum) | | results from different trials have | |
| | extract 63 mg) | | not been consistent. For example, | |
| | and SPE (320 mg) | | studies using the hexanic extract | |
| | once per day | | of Serenoa repens have shown | |
| | for 4 weeks | | improved outcomes among men | |
| | | | with BHP. Hexanic extract has | |
| | | | higher concentrations of the | |
| | | | active ingredients. | |
| | | | P. japonicum extract and saw | |
| | | | palmetto extract may be useful to | |
| | | | decrease frequency of nighttime | |
| | | | urination and residual urine | |
| | | | volume in male patients with | |
| | | | LUTS. | |
| Lifestyle | Fiber supplements | Lifestyle | Many professional urological | [3, 42] |
| changes | (for avoidance or | | associations recommend | |
| | treatment of | | following these measures. | |
| | constipation), | | | |
| | Herbal teas | | | |
| | Timed or organized | | | |
| | voiding (bladder | | | |
| | retraining) | | | |
| | Pelvic floor | | | |
| | exercises | | | |

Negative/avoid recommendations

| | | | | |
|---|---|---|---|---|
| Caloric | Caloric restriction | Diet | Caloric restriction via a low-fat | [43-45] |
| restriction with | either via: | | diet or meal replacement may | |
| and without | Meal replacement- | | alleviate symptoms in obese men | |
| high protein | based low-calorie | | with LUTS | |
| | diet (LCD) ~850-900 | | | |
| | kcal/day (achieved | | | |
| | with 2 meal | | | |
| | replacements/d, | | | |
| | providing a maximum | | | |
| | of 450 kcal of | | | |
| | energy), | | | |
| | or | | | |
| | low-fat, high-protein, | | | |
| | reduced- | | | |
| | carbohydrate (HP) | | | |
| | diet prescribed to | | | |
| | decrease intake by | | | |
| | ~600 kcal/day, | | | |
| | or | | | |
| | reduced calorie | | | |
| | intake by | | | |
| | approximately 2000 | | | |
| | kJ per day via either | | | |
| | reduced fat diet (25- | | | |
| | 30% of total calorie | | | |
| | intake as fat with | | | |
| | <10% saturated fat, | | | |
| | 50-55% as | | | |
| | carbohydrate | | | |
| | and 20% as protein), | | | |
| | or meal replacement | | | |
| | (two sachets daily, | | | |
| | each sachet | | | |
| | providing 160 kcal of | | | |
| | energy, 20 g of | | | |
| | carbohydrate, 14 g of | | | |
| | protein, 3 g of fat) | | | |
| | over 8-12 weeks | | | |
| Caloric | Reduced daily intake | Diet and | Improvements in prostate | [46] |
| restriction and | by ~400 kcal below | lifestyle | symptoms were similar across | |
| exercise | calculated | | both LV and HV groups | |
| | requirement and | | | |
| | randomized to | | | |
| | perform moderate- | | | |
| | intensity exercise of | | | |
| | LV (<150 | | | |
| | minutes/week) or HV | | | |
| | (200-300 | | | |
| | minutes/week) for 24 | | | |
| | weeks | | | |
| Caffeinated, | <2 cups/d of | Diet | Men with chronic prostatitis may | [3, 4] |
| carbonated | caffeinated coffee | | consider avoiding caffeinated and | |
| beverages | <1 cup/day of | | carbonated beverages. | |
| | regular, nondiet | | | |
| | carbonated cola | | | |
| Lifestyle | Drinking less | Lifestyle | Many professional urological | [3, 42, |
| changes | liquids before going | | associations recommend | 47] |
| | to bed, | | following these measures. | |
| | Avoiding spicy | | | |
| | foods, coffee and | | | |
| | caffeinated | | | |
| | beverages, hot | | | |
| | peppers, moderate | | | |
| | alcohol | | | |
| | consumption, | | | |
| | caffeinated tea, | | | |
| | and chili. | | | |

*EFSA, Dietary Reference Values (DRV) 2017

### REFERENCES

1. Ausmees, K., et al., Fermented whey-based product improves the quality of life of males with moderate lower urinary tract symptoms: A randomized double-blind study. PLoS One, 2018. 13(2): p. e0191640.

2. Galeone, C., et al., Onion and garlic intake and the odds of benign prostatic hyperplasia. Urology, 2007. 70(4): p. 672-6.

3. Herati, A. S., et al., Effects of foods and beverages on the symptoms of chronic prostatitis/chronic pelvic pain syndrome. Urology, 2013. 82(6): p. 1376-80.

4. Maserejian, N. N., et al., Intake of caffeinated, carbonated, or citrus beverage types and development of lower urinary tract symptoms in men and women. Am J Epidemiol, 2013. 177(12): p. 1399-410.

5. Yoo, S., et al., Impact of serum 25-OH vitamin D level on lower urinary tract symptoms in men: a step towards reducing overactive bladder. BJU Int, 2018. 122(4): p. 667-672.

6. Espinosa, G., et al., Vitamin D and benign prostatic hyperplasia—a review. Can J Urol, 2013. 20(4): p. 6820-5.

7. Liu, Z. M., et al., Fruit and Vegetable Intake in Relation to Lower Urinary Tract Symptoms and Erectile Dysfunction Among Southern Chinese Elderly Men: A 4-Year Prospective Study of Mr OS Hong Kong. Medicine (Baltimore), 2016. 95(4): p. e2557.

8. Vostalova, J., et al., Use of selenium-silymarin mix reduces lower urinary tract symptoms and prostate specific antigen in men. Phytomedicine, 2013. 21(1): p. 75-81.

9. Morgia, G., et al., Serenoa repens+selenium+lycopene vs tadalafil 5 mg for the treatment of lower urinary tract symptoms secondary to benign prostatic obstruction: a Phase IV, non-inferiority, open-label, clinical study (SPRITE study). BJU Int, 2018. 122(2): p. 317-325.

10. Vidlar, A., et al., Cranberry fruit powder (Flowens) improves lower urinary tract symptoms in men: a double-blind, randomized, placebo-controlled study. World J Urol, 2016. 34(3): p. 419-24.

11. Vidlar, A., et al., The effectiveness of dried cranberries (Vaccinium macrocarpon) in men with lower urinary tract symptoms. Br J Nutr, 2010. 104(8): p. 1181-9.

12. Calogero, A. E., et al., The use of nutraceuticals in male sexual and reproductive disturbances: position statement from the Italian Society of Andrology and Sexual Medicine (SIAMS). J Endocrinol Invest, 2017. 40(12): p. 1389-1397.

13. Schwarz, S., et al., Lycopene inhibits disease progression in patients with benign prostate hyperplasia. J Nutr, 2008. 138(1): p. 49-53.

14. Coulson, S., et al., A phase II randomised double-blind placebo-controlled clinical trial investigating the efficacy and safety of ProstateEZE Max: a herbal medicine preparation for the management of symptoms of benign prostatic hypertrophy. Complement Ther Med, 2013. 21(3): p. 172-9.

15. Ledda, A., et al., Meriva(R), a lecithinized curcumin delivery system, in the control of benign prostatic hyperplasia: a pilot, product evaluation registry study. Panminerva Med, 2012. 54(1 Suppl 4): p. 17-22.

16. Cosentino, V., A. Militello, and G. Lauria, Short-term effects of a dietary supplement on lower urinary tract symptoms. J Biol Regul Homeost Agents, 2018. 32(6): p. 1557-1563.

17. Wong, W. C., et al., Isoflavones in treating watchful waiting benign prostate hyperplasia: a double-blinded, randomized controlled trial. J Altern Complement Med, 2012. 18(1): p. 54-60.

18. Guan, Q., et al., The Effect of Flavonoids on Chronic Prostatitis: A Meta-analysis of Published Randomized Controlled Trials. J Natl Med Assoc, 2019. 111(5): p. 555-562.

19. Shoskes, D. A., et al., Quercetin in men with category III chronic prostatitis: a preliminary prospective, double-blind, placebo-controlled trial. Urology, 1999. 54(6): p. 960-3.

20. Franco, J. V. A., et al., Pharmacological interventions for treating chronic prostatitis/chronic pelvic pain syndrome. Cochrane Database of Systematic Reviews, 2019(10).

21. Maurizi, A., et al., The role of nutraceutical medications in men with non bacterial chronic prostatitis and chronic pelvic pain syndrome: A prospective non blinded study utilizing flower pollen extracts versus bioflavonoids. Arch Ital Urol Androl, 2019. 90(4): p. 260-264.

22. Zhang, W., et al., Effects of dietary flaxseed lignan extract on symptoms of benign prostatic hyperplasia. J Med Food, 2008. 11(2): p. 207-14.

23. Wilt, T. J., et al., Beta-sitosterols for benign prostatic hyperplasia. Cochrane Database of Systematic Reviews, 1999(3).

24. Nabavizadeh, R., et al., Herbal Supplements for Prostate Enlargement: Current State of the Evidence. Urology, 2018. 112: p. 145-150.

25. Ghorbanibirgani, A., A. Khalili, and L. Zamani, The efficacy of stinging nettle (Urtica dioica) in patients with benign prostatic hyperplasia: a randomized double-blind study in 100 patients. Iranian Red Crescent medical journal, 2013. 15(1): p. 9-10.

26. Safarinejad, M. R., Urtica dioica for treatment of benign prostatic hyperplasia: a prospective, randomized, double-blind, placebo-controlled, crossover study. J Herb Pharmacother, 2005. 5(4): p. 1-11.

27. Vahlensieck, W., et al., Effects of pumpkin seed in men with lower urinary tract symptoms due to benign prostatic hyperplasia in the one-year, randomized, placebo-controlled GRANU study. Urol Int, 2015. 94(3): p. 286-95.

28. Leibbrand, M., et al., Effects of an Oil-Free Hydroethanolic Pumpkin Seed Extract on Symptom Frequency and Severity in Men with Benign Prostatic Hyperplasia: A Pilot Study in Humans. J Med Food, 2019. 22(6): p. 551-559.

29. Nemr Elie, et al., Prospective Multicenter Open-Label One-Arm Trial Investigating a Pumpkin Seed, Isoflavonoids, and Cranberry Mix in Lower Urinary Tract Symptoms/Benign Prostatic Hyperplasia: A Pilot Study. Advances in Urology, 2020. 2020 p. 6.

30. Marks, L. S., et al., Effects of a saw palmetto herbal blend in men with symptomatic benign prostatic hyperplasia. J Urol, 2000. 163(5): p. 1451-6.

31. Hong, H., C. S. Kim, and S. Maeng, Effects of pumpkin seed oil and saw palmetto oil in Korean men with symptomatic benign prostatic hyperplasia. Nutr Res Pract, 2009. 3(4): p. 323-7.

32. Wilt, T., et al., Pygeum africanum for benign prostatic hyperplasia. Cochrane Database Syst Rev, 2002(1): p. Cd001044.

33. Cai, T., et al., Effects of pollen extract in association with vitamins (DEPROX 500(R)) for pain relief in patients affected by chronic prostatitis/chronic pelvic pain syndrome: results from a pilot study. Urologia, 2013. 80 Suppl 22: p. 5-10.

34. McNicholas, T. and R. Kirby, Benign prostatic hyperplasia and male lower urinary tract symptoms (LUTS). BMJ Clin Evid, 2011. 2011.

35. Cai, T., et al., The role of flower pollen extract in managing patients affected by chronic prostatitis/chronic pelvic pain syndrome: a comprehensive analysis of all published clinical trials. BMC urology, 2017. 17(1): p. 32-32.

36. Macchione, N., et al., Flower Pollen Extract in Association with Vitamins (Deprox 500(R)) Versus Serenoa repens in Chronic Prostatitis/Chronic Pelvic Pain Syndrome: A Comparative Analysis of Two Different Treatments. Antiinflamm Antiallergy Agents Med Chem, 2019. 18(2): p. 151-161.

37. Bertaccini, A., et al., Observational database Serenoa repens (DOSSER): overview, analysis and results. A multicentric SIUrO (Italian Society of Oncological Urology) project. Arch Ital Urol Androl, 2012. 84(3): p. 117-22.

38. Gravas S, et al., European Association of Urology Guidelines: Management of Non-neurogenic Male LUTS. Edn. presented at the EAU Annual Congress Barcelona 2019. 2019.

39. Kageyama, S., et al., Clinical Effects of Formulated Food of Peucedanum japonicum Extract and Saw Palmetto Extract in Male Patients with Lower Urinary Tract Symptoms. Low Urin Tract Symptoms, 2018. 10(2): p. 167-174.

40. MacDonald, R., et al., Serenoa repens monotherapy for benign prostatic hyperplasia (BPH): an updated Cochrane systematic review. BJU international, 2012. 109(12): p. 1756-1761.

41. Suter, A., et al., Improving BPH symptoms and sexual dysfunctions with a saw palmetto preparation? Results from a pilot trial. Phytother Res, 2013. 27(2): p. 218-26.

42. Nickel, J. C., et al., Canadian Urological Association guideline on male lower urinary tract symptoms/benign prostatic hyperplasia (MLUTS/BPH): 2018 update. Canadian Urological Association Journal, 2018. 12(10).

43. Khoo, J., et al., Comparing effects of a low-energy diet and a high-protein low-fat diet on sexual and endothelial function, urinary tract symptoms, and inflammation in obese diabetic men. J Sex Med, 2011. 8(10): p. 2868-75.

44. Khoo, J., et al., Effects of a low-energy diet on sexual function and lower urinary tract symptoms in obese men. Int J Obes (Lond), 2010. 34(9): p. 1396-403.

45. Khoo, J., et al., Comparing the effects of meal replacements with an isocaloric reduced-fat diet on nutrient intake and lower urinary tract symptoms in obese men. J Hum Nutr Diet, 2014. 27(3): p. 219-26.

46. Khoo, J., et al., Comparing Effects of Low- and High-Volume Moderate-Intensity Exercise on Sexual Function and Testosterone in Obese Men. The Journal of Sexual Medicine, 2013. 10(7): p. 1823-1832.

47. Bradley, C. S., et al., Evidence of the Impact of Diet, Fluid Intake, Caffeine, Alcohol and Tobacco on Lower Urinary Tract Symptoms: A Systematic Review. J Urol, 2017. 198(5): p. 1010-1020.

1. A method of enhancing fertility and conception for individuals with prostate conditions comprising:
requesting and receiving a plurality of user attributes;
comparing the plurality of user attributes to a corresponding plurality of evidence-based fertility benchmarks;
determining a plurality of fertility support opportunities based on at least the plurality of user attributes and the comparison to the corresponding plurality of evidence-based fertility benchmarks;
identifying a plurality of fertility enhancing recommendations based on at least the plurality of fertility support opportunities; and
presenting at least one of the plurality of fertility enhancing recommendations.

2. The method according to claim 1 wherein the individuals are considered to have prostate conditions selected from the group consisting of: prostatitis, benign prostate hyperplasia, inflammation of the prostate, lower urinary tract symptoms (LUTS) and recovery from prostate surgery or radiation.

3. The method according to claim 1, wherein the plurality of user attributes comprises information regarding the prostate conditions and at least one of the user attributes selected from: age, gender, weight, height, activity level, food sensitivities, preferred diet, fertility status, lifestyle choices, and any co-morbidities.

4. The method according to claim 1 wherein the individuals are male and at least 30 years of age or older.

5. The method according to claim 1 wherein the identifying of a plurality of fertility enhancing recommendations includes the dietary recommendations.

6. The method according to claim 1 wherein the identifying of a plurality of fertility enhancing recommendations includes recommendations for supplements.

7-9. (canceled)

10. The method according to claim 1 wherein the recommendation for supplements is to
be administered in the combination of selenium and silymarin for at least 6 months.

11. The method according to claim 1 wherein the recommendation for supplements is to
be administered in the combination of Saw palmetto and Peucedanum japonicum extract for at
least 4 weeks.

12. The method according to claim 1 wherein the recommendation for supplements is to
be administered in the combination of Secale cereale with vitamins selected from the group
consisting of: vitamin B1, vitamin B2, vitamin B3, vitamin B6, folic acid, and vitamin B12.

13. The method according to claim 1 wherein the identifying of a plurality of fertility enhancing recommendations includes food recommendations selected from the group of consisting of:
(i) onions in the amount of at least 4 portions each week for 4 weeks;
(ii) garlic in the amount of at least 4 portions each week for 4 weeks;
(iii) citrus juice in the amount of at least 240 ml per day;
(iv) vegetables and fruits in the amount of at least about 700 g per 2000 kcal diet per day: at least 400 g vegetables of which at least 100 g are dark, leafy vegetables and at least 300 g fruit of which at least 20 g are tomatoes; and
(v) pumpkin seeds in the amount of at least about 5 g/day or pumpkin seed extract in the amount of at least about 500 mg/day.

14. The method according to claim 1 wherein the identifying of a plurality of fertility enhancing recommendations includes diet recommendations selected from the group of:
(i) taking fiber supplements for avoidance or treatment of constipation;
(ii) consuming herbal teas to ensure sufficient daily hydration;
(iii) caloric restriction with and without high protein in obese men with a BMI over 25 kg/m2 to order to lose weight;
(iv) reducing alcoholic beverages and carbonated beverages to less than one per day;
(v) reducing caffeinated beverages to less than two per day; and
(vi) avoiding spicy foods and reducing hot peppers and chili.

15. The method according to claim 1 wherein the identifying of a plurality of fertility enhancing recommendations includes lifestyle recommendations:
(i) timed or organized voiding of bladder to avoid risk of urinary tract infections and
(ii) pelvic floor muscle exercises.

16. The method according to claim 1 for preventing or treating inflammation of the prostate and lower urinary tract symptoms (LUTS).

17. The method according to claim 1 wherein the identifying of a plurality of fertility enhancing recommendations based on at least the plurality of fertility support opportunities comprises:
providing the plurality of fertility support opportunities to a cloud-based artificial intelligence service; and
receiving, from the cloud-based artificial intelligence service, a plurality of fertility enhancing recommendations based on the fertility support opportunities provided to the cloud-based artificial intelligence service.

18. A computer implemented system for generating fertility enhancing recommendations for enhancing fertility and conception for individuals with prostate conditions comprising:
a memory;
a CPU;
a display configured to present an attribute questionnaire to a user;
an attribute receiving unit configured to receive a plurality of user attributes based on at least the attribute questionnaire;
an attribute comparison unit configured to compare the received plurality of user attributes to a corresponding plurality of fertility attribute benchmarks;
an evidence-based diet and lifestyle recommendation engine configured to generate a plurality of fertility enhancing recommendations based on at least the plurality of user attributes and the comparison to corresponding plurality of fertility attribute benchmarks; and
wherein the display is further configured to present at least one of the plurality of the fertility enhancing recommendations to the user.

19. The system of claim 18, wherein the plurality of user attributes comprises information regarding the individual with prostate conditions selected from the group:
prostatitis, benign prostate hyperplasia, inflammation of the prostate, lower urinary tract symptoms (LUTS) or recovery from prostate surgery or radiation.

20. The system according to claim 18 wherein the plurality of user attributes comprises information regarding at least one further user attribute selected from the group of: age, gender, weight, height, activity level, food sensitivities, preferred diet, fertility status, lifestyle choices, and any co-morbidities.

21. (canceled)

22. The system according to claim 18, wherein the evidence-based diet and lifestyle recommendation engine comprises:
a plurality of databases comprising one or more of recipes, specific food items, products, or diet tips;
a plurality of filters by dietary restrictions comprising one or more of food sensitivities, preferred diets, fertility-related conditions, or co-morbidities; and
an optimization unit configured to optimize the plurality of fertility enhancing recommendations based on one or more of caloric intake, food groups, or nutrients.

23. The system according to claim 18 wherein the optimization unit is configured to optimize fertility enhancing recommendations including recommendations of dietary supplements for individuals with prostate conditions, comprising dietary supplements selected from the group of:
(i) Fermented whey protein
(ii) Vitamin D
(iii) Selenium
(iv) Silymarin
(v) Saw palmetto
(vi) Cranberry powder
(vii) Carotenoids selected from: lycopene and beta-carotene
(viii) Curcumin
(ix) Soy isoflavones
(x) Quercetin flavonoid
(xi) Flax lignin extract
(xii) Beta-sitosterols
(xiii) Urica dioica extract
(xiv) Pygeum africanum
and
(xv) Secale cereal, rye pollen extract.

24. The system of claim 18 wherein the optimization unit is configured to optimize fertility enhancing recommendations including recommendations of dietary supplements for individuals with prostate conditions, comprising supplements administered in the following dosing amounts as separate supplements or in combination selected from the group consisting of:
(i) Fermented whey protein in the amount of at least about 200 g/day for at least 4 weeks;
(ii) Vitamin D in the amount of at least about 15 ug/day;
(iii) Selenium in the amount of at least about 240 ug/day for at least 6 months;
(iv) Silymarin in the amount of at least 570 mg/day for at least 6 months;
(v) Saw palmetto in the amount of at least 320 mg/day for at least 72 weeks;
(vi) Cranberry powder in the amount of 250 to 1500 mg/day;
(vii) Carotenoids selected from: lycopene in the amount of at least 15 mg/day for at least 6 months and beta-carotene in the amount of at least 15 mg/day for at least 6 months;
(viii) Curcumin in the amount of 200 to 1000 mg/day for at least 5.5 months;
(ix) Soy isoflavones in the amount of 40 mg/day for at least 12 months;
(x) Quercetin flavonoid in the amount of 500-1000 mg/day for at least 4 weeks;
(xi) Flax lignin extract in the amount of 300-600 mg/day;
(xii) Beta-sitosterols in the amount of 60-195 mg/day for at least 26 weeks;
(xiii) Urica dioica extract in the amount of 120-600 mg/day for at least 6 months;
(xiv) Pygeum africanum in the amount of 75-320 mg/day for at least 84 days; and
(xv) Secale cereal, rye pollen extract in the amount of 500-1000 mg/day.

25-28. (canceled)
